(19) United States
(12) Patent Application Publication
(10) Pub. No.: US 2021/0035064 A1
(43) Pub. Date: Feb. 4, 2021

(54) INFORMATION PROVIDING METHOD AND INFORMATION PROVIDING SYSTEM

(71) Applicant: Panasonic Corporation, Osaka (JP)

(72) Inventors: YURI NISHIKAWA, Kanagawa (JP); JUN OZAWA, Nara (JP)

(21) Appl. No.: 17/073,456

(22) Filed: Oct. 19, 2020

Related U.S. Application Data
(63) Continuation of application No. PCT/JP2019/020657, filed on May 24, 2019.

(30) Foreign Application Priority Data
Jun. 27, 2018 (JP) 2018-122084
Jan. 30, 2019 (JP) 2019-014658

(51) Int. Cl. G06Q 10/08; A61B 5/024; G06Q 10/04; G16H 40/67; G16H 50/30; G01C 21/34; G01C 21/36; G06Q 10/06; G06F 3/0482

(52) U.S. Cl. CPC G06Q 10/08355; A61B 5/024; G06Q 10/047; G16H 40/67; G16H 50/30; G06F 3/0482; G06Q 10/0838; G01C 21/367; G06Q 10/063114; G06Q 10/0833; G01C 21/3407

(57) ABSTRACT

First package information indicating a size or weight of each of packages and first delivery route information indicating first delivery routes are acquired from a memory, where each of the first delivery routes starts from a delivery start point, passes through destinations to which the packages are to be delivered, and ends at the delivery start point. An evaluation value for each of the first delivery routes is calculated based on the first package information and the first delivery route information. An optimum first delivery route is determined, based on the calculated evaluation values, from among the first delivery routes. Information indicating the determined first delivery route is output to an information terminal. The determined first delivery route is displayed on a display of the information terminal. A delivery person delivers the packages to the destinations along the determined first delivery route.

### BACKGROUND

### 1. Technical Field

The present disclosure relates to a technique for presenting, to a delivery person, an optimum delivery route along which the delivery person delivers packages to delivery destinations.

### 2. Description of the Related Art

Japanese Unexamined Patent Application Publication No. 2005-352599 (hereafter referred to as Patent Literature 1) discloses a technique for supporting a delivery person to perform a work safely and smoothly in carrying a package by a delivery vehicle. More specifically, in Patent Literature 1, based on driving caution point information including information indicating locations where delivery persons experienced dangerous situations in the past, a map screen is generated which displays specific content of caution required for each of locations, on a delivery route, where caution is to be paid by a delivery person, and the map screen is presented to the delivery person.

Japanese Unexamined Patent Application Publication No. 2001-76285 (hereinafter referred to as Patent Literature 2) discloses a technique for presenting, to a delivery person, specific stop positions, at which a delivery vehicle may be stopped, associated with delivery destinations where packages are to be delivered and degrees of priority for the respective stop positions, in a delivery operation by a delivery vehicle.

### SUMMARY

However, none of the above-mentioned patent documents takes into consideration a situation in which a delivery person gets off a delivery vehicle and delivers packages on foot, and a physical load on the delivery person in this situation is not taken into consideration at all, and thus an improvement is needed.

One non-limiting and exemplary embodiment provides a technique for accurately calculating a delivery route which enables a delivery person to efficiently deliver packages to destinations on foot with a reduced physical burden on the delivery person.

In one general aspect, the techniques disclosed here feature an information providing method, including, by a computer in an information providing system, acquiring, from a memory, first package information indicating a size or a weight of each of packages and first delivery route information indicating first delivery routes, where each of the first delivery routes starts from a delivery start point, passes through destinations to which the packages are to be delivered, and ends at the delivery start point, calculating, based on the first package information and the first delivery route information, an evaluation value for each of the first delivery routes, determining, based on the calculated evaluation values, an optimum first delivery route from among the first delivery routes, and outputting, to a first information terminal, information indicating the determined first delivery route, wherein the determined first delivery route is displayed on a display of the first information terminal, and wherein a delivery person delivers the packages to the destinations along the determined first delivery route.

The general or specific aspect may be implemented as an apparatus, a system, an integrated circuit, a computer program, a computer-readable storage medium, or any selective combination of an apparatus, a system, a method, an integrated circuit, a computer program, and a computer-readable storage medium. The computer readable storage medium may be, for example, a non-transitory storage medium such as a compact disc-read only memory (CD-ROM), or the like.

According to the present disclosure, when a delivery person delivers packages to destinations on foot, it becomes possible to calculate a delivery route that enables the delivery person to efficiently deliver the packages with a reduced physical load.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a diagram illustrating an example of a network configuration of an information providing system according to Embodiment 1;

FIG. 2 is a block diagram illustrating an example of a configuration of the information providing system illustrated in FIG. 1;

FIG. 3 is a diagram illustrating an example of a data structure of each of a package DB and a customer DB stored in a memory of a server;

FIG. 4 is a diagram illustrating an example of a data structure of each of a package-delivery route DB, a delivery route DB, and a package group DB stored in a memory of a server;

FIG. 5 is a diagram illustrating an example of a data structure of a route segment length DB stored in a memory of a server;

FIG. 6 is a diagram illustrating symbols used to indicate delivery routes stored in the delivery route DB;

FIG. 7 is a sequence diagram illustrating an example of a sequence of data transmission/reception between a server and a delivery person terminal in the information providing system illustrated in FIG. 1;

FIG. 8 is a flowchart illustrating an example of a process performed by an information providing system according to Embodiment 1;

FIG. 9 is a diagram illustrating an example of a screen displaying an optimum delivery route on a delivery person terminal;

FIG. 10 is a diagram illustrating an example of a data structure of a delivery start point DB according to Embodiment 2;

FIG. 11 is a sequence diagram illustrating an example of a sequence of data transmission/reception between a server and a delivery person terminal in an information providing system according to Embodiment 2;

FIG. 12 is a flowchart illustrating an example of a process performed by an information providing system according to Embodiment 2;

FIG. 13 is a sequence diagram illustrating an example of a sequence of data transmission/reception between a server and a delivery person terminal in an information providing system according to Embodiment 3;

FIG. 14 is a flowchart illustrating an example of a process performed by an information providing system according to Embodiment 3;

FIG. 15 is a flowchart illustrating an example of a process performed by an information providing system according to Embodiment 5;

FIG. 16 is a diagram illustrating an example of a data structure of a customer DB according to Embodiment 5;

FIG. 17 is a flowchart illustrating an example of a process performed by the information providing system according to Embodiment 5;

FIG. 18 is a diagram illustrating an example of a display screen displayed on a delivery person terminal according to Embodiment 6;

FIG. 19 is a flowchart illustrating an example of a process performed by an information providing system according to Embodiment 6;

FIG. 20 is a diagram illustrating an example of a data structure of a package group DB stored in a memory of a server according to Embodiment 7;

FIG. 21 is a flowchart illustrating an example of a process performed by an information providing system according to Embodiment 7;

FIG. 22 is a diagram illustrating an example of a data structure of a package group-delivery start point DB stored in a memory of a server according to Embodiment 8;

FIG. 23 is a flowchart illustrating an example of a process performed by an information providing system according to Embodiment 8;

FIG. 24 is a flowchart illustrating details of a process of a delivery start point evaluation subroutine in S72 in FIG. 23;

FIG. 25 is a diagram illustrating an example of a reason registration screen displayed on a delivery person terminal according to Embodiment 9;

FIG. 26 is a diagram illustrating an example of a data structure of a package group-delivery start point DB stored in a memory of a server according to Embodiment 9;

FIG. 27 is a flowchart illustrating an example of a process of a delivery start point evaluation subroutine according to Embodiment 9;

FIG. 28 is a diagram illustrating an example of an evaluation registration screen displayed on a delivery person terminal according to Embodiment 10;

FIG. 29 is a diagram illustrating an example of a data structure of a package group-delivery start point DB stored in a memory of a server according to Embodiment 10;

FIG. 30 is a flowchart illustrating an example a process of a delivery start point evaluation subroutine according to Embodiment 10;

FIG. 31 is a diagram illustrating an example of a network configuration of an information providing system according to Embodiment 11;

FIG. 32 is a diagram illustrating an example of a data structure of a weather distance DB stored in a memory of a server according to Embodiment 11;

FIG. 33 is a flowchart illustrating an example of a process performed by the information providing system according to Embodiment 11;

FIG. 34 is a diagram showing a formula for calculating an evaluation value;

FIG. 35 is a diagram illustrating an example of a data structure of a size-dependent load coefficient DB stored in a memory of a server according to Embodiment 13;

FIG. 36 is a diagram illustrating an example of a data structure of a type-dependent load coefficient DB stored in a memory of a server according to Embodiment 14;

FIG. 37 is a diagram illustrating an example of a data structure of an elevation difference-dependent load coefficient DB stored in a memory of a server according to Embodiment 15;

FIG. 38 is a diagram illustrating an example of a data structure of each of two route segment length DBs stored in a memory of a server according to Embodiment 16;

FIG. 39 is a diagram illustrating an example of a data structure of a heart rate-dependent load coefficient DB stored in a memory of a server according to Embodiment 16;

FIG. 40 is a diagram illustrating an example of a data structure of each of an upper limit DB and a delivery person DB stored in a memory of a server according to Embodiment 17;

FIG. 41 is a flowchart illustrating an example of a process performed by an information providing system according to Embodiment 17;

FIG. 42 is a diagram illustrating an example of a delivery route;

FIG. 43 is a probability binary tree representation of the delivery route illustrated in FIG. 42;

FIG. 44 is a diagram further illustrating the binary tree illustrated in FIG. 43;

FIG. 45 is a diagram further illustrating the binary tree illustrated in FIG. 44;

FIG. 46 is a diagram further illustrating the binary tree illustrated in FIG. 45;

FIG. 47 is a diagram further illustrating the binary tree illustrated in FIG. 46;

FIG. 48 is a diagram further illustrating the binary tree illustrated in FIG. 47;

FIG. 49 is a probability binary tree representation of a delivery route different from the delivery route illustrated in FIG. 42;

FIG. 50 is a diagram illustrating an example of a data structure of a size-dependent load coefficient DB stored in a memory of a server according to Embodiment 18;

FIG. 51 is a diagram illustrating an example of a data structure of a type-dependent load coefficient DB stored in a memory of a server according to Embodiment 18;

FIG. 52 is a diagram illustrating an example of a data structure of an elevation difference-dependent load coefficient DB stored in a memory of a server according to Embodiment 18;

FIG. 53 is a diagram illustrating an example of a data structure of a route segment length DB according to Embodiment 18;

FIG. 54 is a flowchart illustrating an example of a process performed by an information providing system according to Embodiment 18;

FIG. 55 is a diagram illustrating an example of a network configuration of an information providing system according to Embodiment 19; and

FIG. 56 is a diagram illustrating an example of a notification screen displayed on a user terminal to inform that a delivery vehicle is moving to a recipient according to Embodiment 19.

### DETAILED DESCRIPTION

### Underlying Knowledge Forming Basis of the Present Disclosure

Recently, the logistics industry is facing a serious shortage of human resources. In particular, there is a serious shortage of delivery persons who drive delivery vehicles to deliver packages to destinations. Thus, a large number of new delivery persons will be employed. Therefore, it is desired to develop a tool for enabling even new delivery persons to perform an efficient delivery operation as skilled delivery persons.

A skilled delivery person has knowledge and know-how as to (1) where a delivery vehicle is to be stopped when packages are to be carried on foot from the sopped place, (2) how packages are to be grouped at the stopped place and in what order the packages are to be carried, and (3) time zones in which recipients are likely to be at home at various destinations. By making full use of such knowledge and know-how, the skilled delivery person achieves a high throughput. Note that throughput may be defined by the number of deliveries completed per unit time.

As described above, a skilled delivery person has a high knowledge in a situation where a delivery vehicle is stopped and packages are delivered on foot to destinations near a delivery start point such that a high throughput is achieved.

Therefore, in order to improve the throughput while lowering the training cost for new employees, it is effective to use a scheduler that determines an optimum delivery route in a situation where packages are carried on foot after a delivery vehicle is stopped.

For this purpose, it is necessary to calculate the delivery route taking into account not only a travel distance but also package information as to the size or weight of packages actually carried by the delivery person.

In the above situation, the optimum delivery route may be calculated further taking into account a physical load on the delivery person such that a further reduction in the physical load on the delivery person is achieved.

The techniques disclosed in Patent Literature 1 and Patent Literature 2 described above are both related to the technique of calculating the delivery route taken by the delivery vehicle, and are not the technique of calculating the delivery route to be taken by the delivery person after getting off the delivery vehicle, and thus these techniques cannot not be applied to the situations handled by the present disclosure. In both techniques disclosed in Patent Literature 1 and Patent Literature 2, neither the package weight nor the package size is taken into account in calculating the delivery route, and these techniques do not allow it to calculate the optimum delivery route in the situations handled by the present disclosure. Since Patent Literature 1 and Patent Literature 2 do not consider the situations handled by the present disclosure, they cannot determine a delivery route provides a reduced physical load on the delivery person.

The present disclosure provides a technique for accurately calculating a delivery route which allows a delivery person to efficiently deliver packages to destinations on foot with a reduced physical burden on the delivery person.

According to an aspect, the present disclosure provides an information providing method including,

by a computer in an information providing system,

acquiring, from a memory, first package information indicating a size or a weight of each of packages and first delivery route information indicating first delivery routes, wherein each of the first delivery routes starts from a delivery start point and passes through destinations to which the packages are to be delivered and ends at the delivery start point,

calculating, based on the first package information and the first delivery route information, an evaluation value for each of the first delivery routes,

determining, based on the calculated evaluation values, an optimum first delivery route from among the first delivery routes, and

outputting, to a first information terminal, information indicating the determined first delivery route,

wherein the determined first delivery route is displayed on a display of the first information terminal, and

wherein a delivery person delivers the packages to the destinations along the determined first delivery route.

In the information providing method according to the present aspect, the optimum first delivery route is determined based on the evaluation values calculated using the first package information indicating the size or weight of each of the packages. That is, the delivery route determined according to the present aspect is a low-load delivery route that allows the delivery person to efficiently deliver packages on foot from the delivery start point.

The first delivery route determined in the above-described manner is displayed on the information terminal of the delivery person, which allows the delivery person to efficiently deliver packages on foot. Therefore, even a new delivery person can deliver packages with high efficiency similar to that with which an experienced delivery person can.

Furthermore, in this aspect, the optimum first delivery route is determined based on the evaluation values calculated using the first package information regarding the size or weight of each of the packages to be delivered by the delivery person on foot, which makes it possible to reduce the physical load on the delivery person.

In the method described above, each evaluation value may represent a physical load on the delivery person.

Since the physical load on the delivery person is taken into account in the evaluation values, use of the evaluation values makes it possible to determine the first delivery route that allows a reduction in the physical load on the delivery person.

In the method described above, each evaluation value may reflect a fact that each time the delivery person delivers a package to a destination, the sum of sizes or weights of remaining ones of the packages becomes smaller.

In this method, each time a package is delivered to a destination, the sum of sizes or weights of the remaining packages is reduced and this is taken into account in the calculation of the evaluation values. Thus, it is possible to calculate the evaluation values such that the physical load on the delivery person is accurately considered.

In the method described above, the packages may include a first package, the destinations may include a first destination, and when the delivery person delivers the first package to the first destination, the evaluation value may be recalculated, wherein the recalculated evaluation value may be based on information obtained by removing the size or weight of the first package from the first package information.

In the method described above, each evaluation value is calculated as a sum total of delivery loads on route segments including route segments connecting the delivery start point and the destinations when the delivery start point and the destinations are connected serially and route segments between the destinations,

wherein the delivery load on an i-th route segment (i is an integer equal to or greater 0) may be represented by a product of a package load and a route segment load, where the package load corresponds to weights of one or more packages to be delivered on the i-th route segment and the route segment load corresponds to a distance or a traveling time of the i-th route segment.

In this method, the evaluation values are calculated taking into account the route segment load depending on the distance or travel time of each route segment forming the first delivery route and the weights of the packages carried along each route, the actual burden on the delivery person, and thus the actual load on the delivery person is properly reflected in the calculated evaluation values.

In the method described above, the evaluation values may include an evaluation value of any first delivery route P included in the first delivery routes. When the delivery start point on the first delivery route P is denoted by D0, and the destinations on the first delivery route P are denoted by D1 to Dn, let route segments on this first delivery route P denoted as follows: a route segment starting at D0 and ending at D1 as a 0-th route segment; a route segment starting at D1 and ending at D2 as a 1st route segment; a route segment starting at Di and ending at D(i+1) as an i-th route segment; and a route segment starting at Dn and ending at D(n+1) as an n-th route segment, where D(n+1) is identical to D0, then (the evaluation value for the first delivery route)=(the delivery load on the 0th route segment)+ . . . +(the delivery load on the i-th route segment)+ . . . +(the delivery load on the n-th route segment), (the delivery load on the i-th route segment)=(the package load on the i-th route segment)×(the route segment load on the i-th route segment), the package load on the i-th route segment has a value depending on the weights packages to be delivered between Di and D(i+1), and the route segment load on the i-th route segment has a value depending on the distance of the i-th route segment or the i-th travel time taken by the delivery person to travel the i-th route segment, where 0≤i≤n (i and n are each an integer).

In this method, the first delivery route information may include at least one of the followings: distances of the first delivery routes; travel times of the first delivery routes; and road conditions of the first delivery routes.

In this method, since the first delivery route information includes at least one of the followings: distances of the first delivery routes; travel times of the first delivery routes; and road conditions of the first delivery routes, the evaluation values of the first delivery routes can be calculated more accurately.

In the method described above, the first delivery route information may includes at least one of (i) to (iii) described below: (i) the distance of the 0th route, . . . , the distance of the n-th route; (ii) the travel time taken by the delivery person on the 0th route segment length of the 0th route, . . . , the travel time taken by the delivery person on the 0th route segment length of the n-th route segment; and (iii) the road condition of the 0th route segment, . . . , the road condition of the n-th route segment.

In the method described above, the package load may be represented by a sum total of values each of which is obtained by multiplying a weight of each package delivered by the delivery person on the i-th route segment and a first load coefficient of the package, and

the first load coefficient may be set so as to increase as the size of the package increases.

In this method, the evaluation value increases as the size of the package increases, and thus the physical load on the delivery person is more properly taken into account in the calculation of the evaluation value.

In the method described above, when packages to be delivered between Di and D(i+1) are denoted as a 1st package, . . . , an m-th package, a weight of the 1st package is denoted as W, . . . , a weight of the m-th package is denoted as Wm, the first load coefficient corresponding to the 1st load is denoted as β1, . . . , and the first load coefficient corresponding to the m-th load is denoted as βm, then

the package load on the i-th route segment may be given as the package load on the i-th route segment=(W1×β1)+ . . . +(Wm×βm), where when βj>βk, the size of the j-th package>the size of the k-th package.

In the method described above, the package load may vary depending on whether or not a trolley is used.

In this method, the package load varies depending on whether or not a trolley is used, and thus the evaluation values are calculated taking into account whether the delivery person can use the trolley in delivering the packages.

In the method described above, the first delivery route information may include information regarding whether the trolley is usable.

In this method, the first delivery route information includes information regarding whether the trolley is usable, and thus the delivery person easily grasps whether or not the trolley can be used on the first delivery route, which makes it possible to perform delivering efficiently.

In the method described above, the first delivery route information may include a first route segment on which the delivery person uses the trolley and a second route segment on which the delivery person does not use the trolley.

In this method, since the first delivery route information includes the first route segment on which the delivery person uses the trolley and the second route segment on which the delivery person does not use the trolley, the delivery person can easily distinguish between route segments where the trolley can be used and route segments where the trolley cannot be used, which makes it possible to perform delivering efficiently.

In the method described above, the package load may be represented by a sum total of values each of which is obtained by multiplying a weight of each package carried by the delivery person on the i-th route segment and a second load coefficient of the package, and

the second load coefficient may set to a value according to a type of the package.

In this method, since the evaluation values are calculated taking into account the types of packages, it is possible to calculate the evaluation values such that an actual burden on the delivery person is more appropriately considered.

In the method described above, when packages to be delivered between Di and D(i+1) are denoted as a 1st package, . . . , an m-th package, a weight of the 1st package is denoted as W1, . . . , a weight of the m-th package is denoted as Wm, the second load coefficient corresponding to the 1st package is denoted as γ1, . . . , the second load coefficient corresponding to the m-th package is denoted as γm, . . . , then the package load on the i-th route segment may be given by (W1×γ1) + . . . +(Wm×γm), each of the second load coefficients γ1 . . . , γm may have a value depending on the type of a corresponding package.

In the method described above, the first delivery route information may include a distance of each of the route segments and an elevation difference of each of the route segments,

the route segment load may be represented by a sum total of values each of which is obtained by multiplying the distance of the i-th route segment and a third load coefficient, and

the third load coefficient may be set so as to increase as the elevation difference increases in an uphill direction.

In this method, since the evaluation value of the first delivery route increases with increasing number of route segments in which elevation differences increase in the uphill direction and with increasing elevation differences, it is possible to properly take into account the actual burden on the delivery person in the calculation of the evaluation value.

In the method described above, the first delivery route information may include a length L0 and an elevation difference of the 0th route segment, . . . , a length Ln and an elevation difference of the n-th route segment, when the third load coefficient corresponding to the 0th route segment is denoted as δ0, . . . , the third load coefficient corresponding to the n-th route segment is denoted as δn, then (the route segment load on the 0th route segment)=(L0×δ0), . . . , (the route segment load on the n-th route segment)=(Ln×δn), where if δj>δk, then (the elevation difference of the j-th route segment)>(the elevation difference of the k-th route segment), and (the elevation difference of the j-th route segment)={(the elevation of D(j+1))−(the elevation of Dj)}.

In the method described above, the first delivery route information may include a length of each of the route segments and an average increase rate of a heart rate of the delivery person on each of the route segments,

the route segment load may be represented by a sum total of values each of which is obtained by multiplying the length of an i-th route segment and a fourth load coefficient, and

the fourth load coefficient may be set so as to increase as the average increase rate of the heart rate increases.

In this method, since the evaluation value of the first delivery route increases with increasing number of route segments in which a large average increase rate of the heart rate occurs and with increasing the average increase rate of the heart rate, it is possible to properly take into account the actual burden on the delivery person in the calculation of the evaluation value.

In the method described above, the first delivery route information may include the length L0 of the 0th route segment and the average increase rate of the heart rate of the delivery person on the 0th route segment, . . . , the length Ln of the n-th route segment and the average increase rate of the heart rate of the delivery person on the n-th route segment, wherein when the fourth load coefficient corresponding to the 0th route segment is denoted as ε0, . . . , the fourth load coefficient corresponding to the n-th route segment is denoted as εn, then (the route segment load on the 0th route segment)=(L0×ε0, . . . , (the route segment load on the n-th route segment)=(Ln×εn, where if εj>εk, then (the average increase rate of the heart rate of the delivery person on the j-th route segment)>(the average increase rate of the heart rate of the delivery person on the k-th route segment).

In the method described above, the first package information may include a delivery destination of each of the packages,

delivery destinations whose distances from the delivery start point are equal to or smaller than a threshold value may be extracted, based on the first package information, from the delivery destinations, and

each evaluation value may be calculated for each of the first delivery routes including the extracted delivery destinations as the destinations.

In this method, evaluation values are calculated for the delivery routes including delivery destinations with distances from the delivery start point equal to or smaller than the threshold value, and the optimum delivery route is calculated using the calculated evaluation values. Thus, it is possible to prevent a delivery destination, whose distance from the delivery start point is larger than the threshold value and thus which is difficult to get to on foot, from being set as a destination.

The method described above may further including acquiring, from the memory, delivery start point information in which candidates for the delivery start point are stored in association with positions,

further acquiring a current position of the delivery person, and

further determining, as the delivery start point, a candidate for the delivery start point closest to the current position of the delivery person from among the candidates for the delivery start point.

In this method, among candidates for the delivery start point, a candidate for the delivery start point located closest to the current position of the delivery person is determined as the delivery start point, and thus the delivery person can start delivering the packages on foot from an appropriate delivery start point near the current position even when the delivery person does not know the delivery start point.

In the method described above, in a case where information indicating that the delivery vehicle of the delivery person is unable to be stopped at the delivery start point is acquired from the first information terminal via a network, a candidate for the delivery start point next closest to the current position of the delivery person may be determined as the delivery start point from among the candidates for the delivery start point.

In this method, in the case where the delivery vehicle cannot be stopped at the delivery start point, a delivery start point whose distance from the current position is the second closest is employed, and thus the delivery person is allowed to stop the delivery vehicle at the easy-to-stop delivery start point which allows it to efficiently delivery packages.

In the method described above, the information indicating that the delivery vehicle of the delivery person is unable to be stopped at the delivery start point may include a reason why the delivery vehicle is unable to be stopped.

In this method, when the delivery vehicle cannot be stopped, information indicating the reason therefor is transmitted from the delivery person terminal. Thus it is possible to collect information for determining whether the point is a proper delivery start point.

In the method described above, when the delivery vehicle of the delivery person is enabled to be stopped at the delivery start point, information regarding appropriateness of the delivery start point may be further acquired from the first information terminal via a network.

In this method, in the case where the delivery vehicle of the delivery person can be stopped at the delivery start point, information regarding appropriateness of the delivery start point may be further acquired from the first information terminal via a network.

The method described above may further include,

in a case where, when the delivery person visits a first destination included in the destinations, information indicating that a first recipient at the first destination is absent is acquired from the first information terminal via a network,

acquiring, from the memory, two or more pieces of second delivery route information indicating second delivery routes each of which serially connects remaining destinations starting from the first destination and returns to the delivery start point,

calculating, based on the first package information and the two or more pieces of second delivery route information, an evaluation value for each of the second delivery routes,

determining, based on the calculated evaluation values, an optimum second delivery route from among the second delivery routes,

outputting information indicating the determined second delivery route to the first information terminal, and

causing the second delivery route to be displayed on the display of the first information terminal.

In this method, when the first recipient of the package is absent at the first destination, the optimum second delivery route connecting the first destination to the remaining destinations in order is rescheduled, and thus the optimum second delivery route is determined taking into account the weights or the sizes of the undelivered packages.

The method described above may further include, reading, from the memory, history information indicating a redelivery rate in the past at each of the destinations, and the evaluation value of each of the first delivery routes may be calculated based on the history information.

In this method, since the evaluation value is calculated taking into account the history information indicating the redelivery rate in the past, it is possible to calculate the evaluation values such that the actual situations are more properly reflected. Thus, it is possible to achieve enhanced reliability in the finally determined delivery route.

The method described above may further include

in a case where information is acquired from first information terminal via a network, the information indicating that when the delivery person visits a first destination included in the destinations, a first package included in the packages is delivered and a second package is picked up,

acquiring second package information regarding a size or a weight of the second package from the first information terminal via a network,

acquiring, from the memory, two or more pieces of second delivery route information indicating second delivery routes each of which serially connects remaining destinations starting from the first destination and returns to the delivery start point,

calculating, based on the first package information, the second package information, and the two or more pieces of second delivery route information, an evaluation value for each of the second delivery routes,

determining, based on the calculated evaluation values, an optimum second delivery route from among the second delivery routes,

outputting information indicating the determined second delivery route to the first information terminal, and

causing the second delivery route to be displayed on the display of the first information terminal.

In this method, when the first package is delivered, if another second package is picked up, the second delivery route connecting the first destination to the remaining destinations in order is rescheduled, taking into account the size or the weight of the second package, and thus the optimum delivery route is determined taking into account the weight or size of the picked-up package.

The method described above may further include

in a case where information is stored in advance in the memory, the information indicating that at a first destination included in the destinations, the delivery person is to deliver a first package included in the packages and pick up a second package,

acquiring, from the memory, second package information regarding a size or a weight of the second package,

wherein the evaluation value of each of the first delivery routes is calculated based on the first package information and the second package information.

In this method, when the sizes or weights of packages to be picked up are known in advance, the optimum delivery route is determined taking into account not only the first packages to be delivered but also the second packages to be picked up. As a result, the first delivery route is determined that allows the packages to be delivered with higher efficiency.

The method described above may further include

acquiring, from the first information terminal, information indicating that the delivery person gets off a delivery vehicle,

outputting, to a second information terminal owned by a recipient at a first destination included in the destinations, information indicating that the delivery person is heading to the first destination, and

causing the information indicating that the delivery person is heading to the first destination to be displayed on a display of the second information terminal.

In this method, when the delivery person gets off the delivery vehicle, information is transmitted to the second information terminal of the package recipient to notify that the delivery person is heading to the recipient, which further ensures that the recipient can receive the package.

In the method described above, the first delivery route information may include a distance of each of the first delivery routes, and

the method may further include

acquiring, from the memory, an upper limit length information including a first upper limit distance in walking of the delivery person and a second upper limit distance smaller than the first upper limit distance,

in a case where information indicating bad weather is acquired via a network, setting the second upper limit distance, and

in a case where a distance of the determined first delivery route is equal to or more than the second upper limit distance, deleting a destination included in the first delivery routes such that the distance of the first delivery route becomes smaller than the second upper limit distance.

In this method, in the case of bad weather, the second upper limit of the distance shorter than the first upper limit of the distance is selected.

In a case where the length of the first delivery route is equal to or larger than the second upper limit of the length, a destination is deleted such that the resultant length of the first delivery route becomes smaller than the second upper limit of the length. Thus, an increase in the burden on the delivery person due to a bad weather can be suppressed, and the safety for the delivery person can be ensured.

The method described above may further include

acquiring, from the memory, an upper limit evaluation value corresponding to an age or gender of the delivery person, and

in a case where the evaluation value of the determined first delivery route is equal to or higher than the upper limit evaluation value, deleting a destination included in the first delivery route such that the evaluation value of the first delivery route becomes smaller than the upper limit evaluation value.

In this method, the upper limit of the evaluation value is selected depending on the age and the gender, and in the case where the evaluation value of the first delivery route is equal to or larger than the upper limit of the evaluation value, a destination is deleted such that the resultant evaluation value of the first delivery route becomes smaller than the upper limit of the evaluation value. Thus, the delivery route that is proper in terms of the load on the delivery person is presented depending on the age or the gender of the delivery person.

The present disclosure can also be realized in the form of a computer program that causes a computer to execute steps featuring the method of the present disclosure, or in the form of a system that operates according to the computer program. As a matter of course, such a computer program can be distributed via a computer-readable non-transitory storage medium such as a CD-ROM or a communication network such as the Internet.

Note that each of the embodiments described below shows a specific example of the present disclosure. In the following embodiments of the present disclosure, values, shapes, constituent elements, steps, the order of steps, and the like are described by way of example but not limitation. Among constituent elements described in the following embodiments, those constituent elements that are not described in independent claims indicating highest-level concepts of the present disclosure are optional. Also note that various combinations of part or all of embodiments are possible.

### Embodiment 1

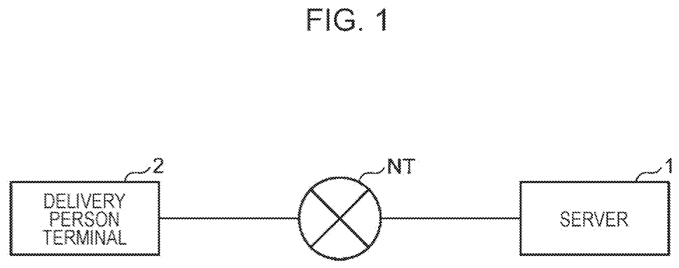

FIG. 1 is a diagram illustrating an example of a network configuration of an information providing system according to Embodiment 1. This information providing system presents an appropriate delivery route to a delivery person such that the delivery person is allowed to deliver packages to destinations on foot according to the presented delivery route. The information providing system includes a server 1 and a delivery person terminal 2 (an example of the first information terminal). The server 1 and the delivery person terminal 2 are connected to each other via a network NT such that they can communicate with each other. As the network NT, for example, an Internet communication network, a mobile phone communication network, and/or the like are used.

The server 1 includes, for example, one or more computers, and performs entire control on the information providing system. The delivery person terminal 2 is realized using a portable information processing apparatus such as a smartphone, a tablet terminal, or the like. The delivery person terminal 2 displays various messages presented to the delivery person who delivers packages. Note that the delivery person terminal 2 may be realized using an information processing apparatus installed on a delivery vehicle used by the delivery person. For example, the delivery person terminal 2 may be implemented in an ECU (Electronic Control Unit) or a car navigation system provided on the delivery vehicle. Alternatively, the delivery person terminal 2 may be realized using a dedicated portable information processing apparatus developed for use by a package delivery person.

Although only one delivery person terminal 2 is illustrated in FIG. 1 for convenience of description, the system may include two or more delivery person terminals 2. In this case, the data transmitted from the delivery person terminal 2 is managed for each delivery person using the delivery person ID individually assigned to the delivery person terminal 2.

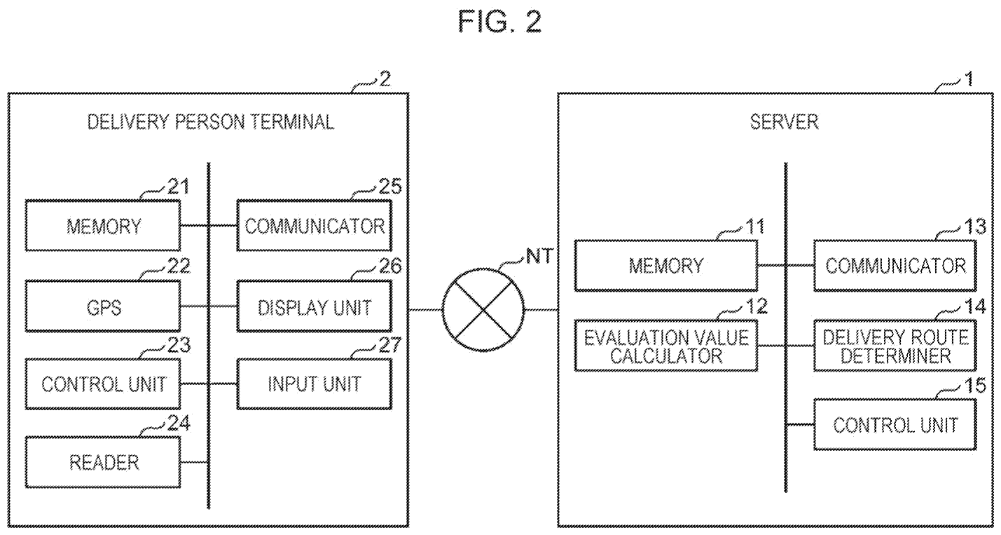

FIG. 2 is a diagram illustrating an example of a configuration of the information providing system illustrated in FIG. 1. The server 1 includes a memory 11, an evaluation value calculator 12, a communicator 13, a delivery route determiner 14, and a control unit 15. The evaluation value calculator 12, the delivery route determiner 14, and the control unit 15 may be implemented by a processor such as a CPU, or may be implemented by a dedicated hardware circuit. In the implementation of these components, each of them may be implemented by separate hardware or may be implemented by a single processor executing a predetermined program.

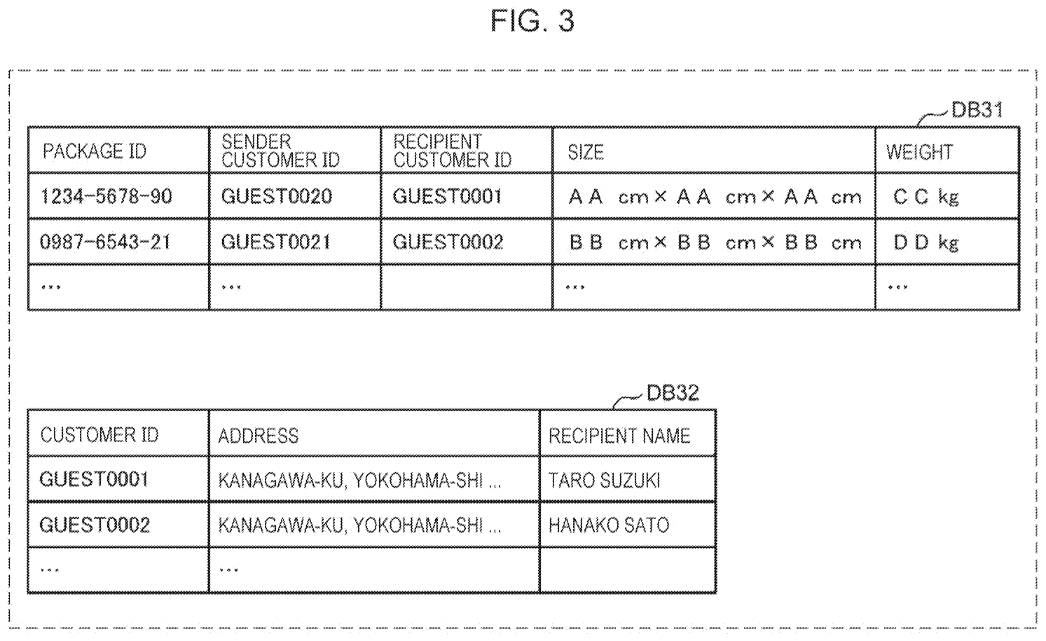

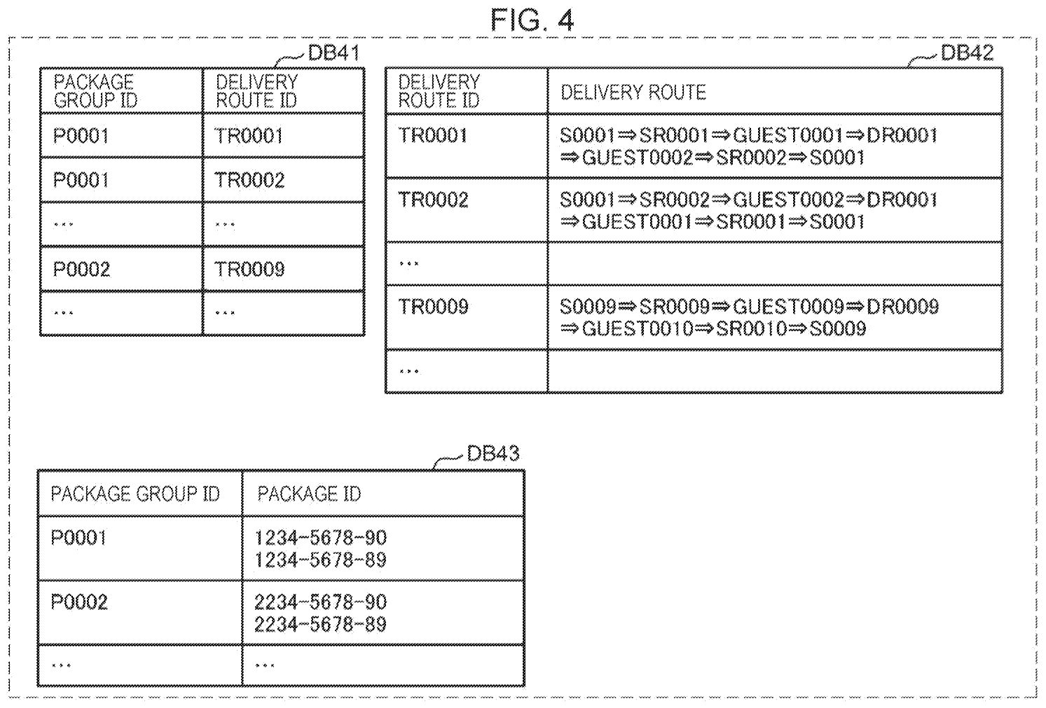

The memory 11 is realized by, for example, a semiconductor memory. The memory 11 stores, in advance, package information (an example of the first package information) regarding the size or weight of each of packages to be delivered by a delivery person from a delivery start point to destinations. The memory 11 stores, in advance, pieces of delivery route information (an example of first delivery route information) indicating delivery routes (example of the first delivery routes) connecting destinations in series starting from the delivery start point. The package information is described in, for example, a package DB 31 which will be explained later with reference to FIG. 3. The pieces of delivery route information are described in, for example, a delivery route DB 42 which will be explained later with reference to FIG. 4.

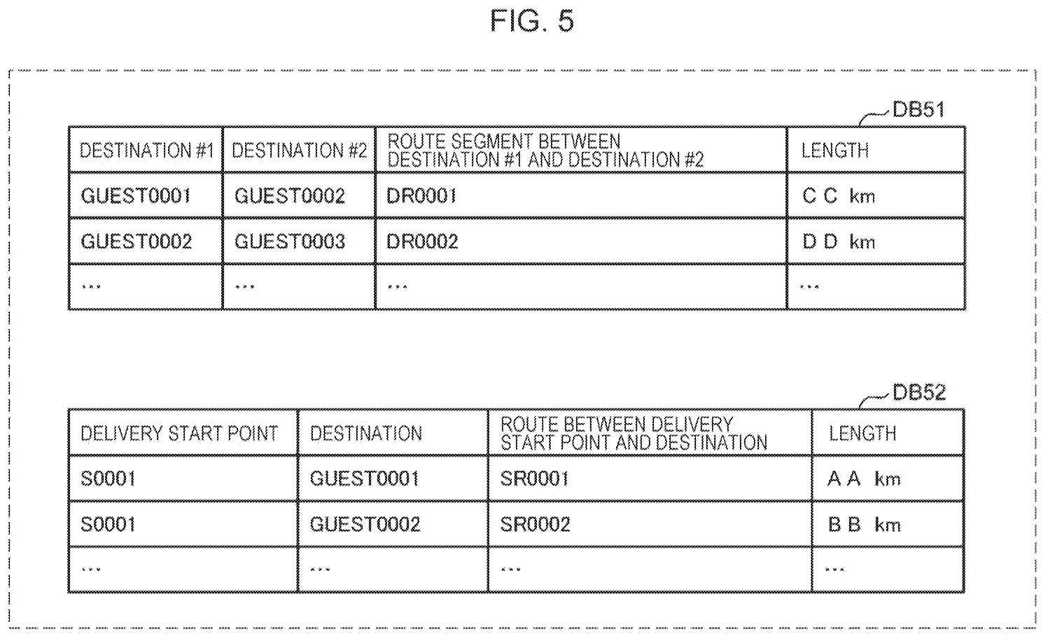

The evaluation value calculator 12 calculates an evaluation value of each of the delivery routes based on the package information and the delivery route information. Each piece of delivery route information includes length information indicating the length of each delivery route. The length information of each delivery route is given, for example, by a route segment length DB 51 and a route segment length DB 52 which will be described later with reference to FIG. 5. The evaluation value is a value for evaluating a physical load on a delivery person when the delivery person carries a package. The larger the load, the larger the evaluation value. Therefore, when the evaluation value of the delivery route is smaller, the delivery person can carry the package more easily, and the physical load on the delivery person is smaller. Thus, based on the evaluation value, it is possible to calculate a delivery route that can result in a reduction in the physical load on the delivery person.

In the present embodiment, the evaluation value calculator 12 calculates the evaluation values taking into the fact that each time a package is delivered to a destination, the sum of sizes or weights of the remaining packages decreases. This allows it to calculate the evaluation value properly taking into account the physical load on the delivery person.

For example, the evaluation value of a delivery route starting from a delivery start point and passes through destinations and finally returning to the delivery start point is represented by a sum total of delivery loads on route segments connecting between the delivery start point and a destination or between two destinations.

The delivery load is represented by the product of a package load and a route segment load where the package load for an i-th route segment is given by a value corresponding to the weight or the size of one or more packages carried along the i-th route segment, while the route segment load is given by a value corresponding to the distance of the i-th route segment or the time (traveling time) needed to move along the i-th route segment and where i is an index (an integer greater than 0) indicating a specific route segment). The weight or size of a package is described in the package DB 31. The value of the route segment load increases as the distance or the required time increases. The distance of the i-th route segment is identified by the route segment length DB 51 and DB 52. The time required for the i-th route segment is calculated by dividing the distance of the i-th route segment by the moving speed of the delivery person on foot. Details of the calculation of the evaluation value according to the embodiment will be described later.

The delivery route delivery route determiner 14 determines an optimum delivery route from the delivery routes based on evaluation values of the respective delivery routes calculated by the evaluation value calculator 12. More specifically, the delivery route having the smallest evaluation value is determined as the optimum delivery route.

The communicator 13 is implemented by a communication apparatus for connecting the server 1 to the network NT. Using the communicator 13, the delivery route determiner 14 transmits information indicating the optimum delivery route determined by the delivery route determiner to the delivery person terminal 2. The control unit 15 controls the entire server 1.

The delivery person terminal 2 includes a memory 21, a GPS 22, a control unit 23, a reader 24, a communicator 25, a display unit 26, and an input unit 27. The memory 21 includes, for example, a semiconductor memory, and stores an application or the like for displaying information indicating a delivery route or the like transmitted from the server 1.

The GPS (Global Positioning System sensor) 22 calculates the present position of the delivery person terminal 2 by using radio waves from a GPS satellite. The GPS 22 may calculate the current position at predetermined time intervals (for example, 1 minute, 2 minutes, 10 minutes, etc.).

The reader 24 is realized, for example, using a bar code reader for reading a bar code or a QR code (registered trademark) described on a package slip attached to a package. The bar code or the QR code (registered trademark) includes at least a package ID which is an identifier of the package.

The reader 24 is used, for example, to read a bar code or a QR code (registered trademark) written on a package slip of a package when a delivery person loads the package into a delivery vehicle at a delivery center. Thus, the delivery person terminal 2 acquires information on the destination (the delivery destination) and the recipient or the like from the server 1 by using the package ID read via the reader 24 as a key, and manages the package to be delivered based on the acquired information.

The reader 24 is also used, for example, to read a bar code or a QR code (registered trademark) written on a package slip when a delivery person delivers the package to a user. This makes it possible for the server 1 to manage whether or not the delivery of the package is completed.

The communicator 25 is realized using a communication apparatus for connecting the delivery person terminal 2 to the network NT, and receives information indicating an optimum delivery route or the like transmitted from the server 1. The communicator 25 also transmits a current position detected by the GPS 22 to the server 1.

The display unit 26 is realized using a display apparatus such as a liquid crystal display, and displays various images including information indicating an optimum delivery route transmitted from the server 1. The input unit 27 is realized using, for example, a touch panel, and accepts various operations performed by a user. The control unit 23 is realized using a processor such as a CPU, and performs overall control of the delivery person terminal 2.

FIG. 3 is a diagram illustrating an example of a data structure of each of a package DB 31 and a customer DB 32 stored in the memory 11 of the server 1. The package DB 31 is a database for storing package information related to packages delivered by a delivery person in which one record is assigned to one package. In the package DB 31, a “package ID”, a “sender customer ID”, a “recipient customer ID”, a “size”, and a “weight” are stored in association with each other.

The “package ID” is an identifier assigned to each package so as to uniquely identify the package. The “sender customer ID” is an identifier identifying a customer who is the sender of the package. The “recipient customer ID” is an identifier identifying a customer who is to receive the package. The “size” indicates the size of the package. Note that the size of the package is given by the product of the width, the height and the depth of the package. Thus, the larger the product value, the larger the size of the package. The weight” indicates the weight of the package.

The customer DB 32 is a database for storing personal information of a customer who is to receive a package, and one record is assigned to one customer. In the customer DB 32, the “customer ID” an “address”, and a “recipient Name” are stored in association with each other. The “address” indicates the address of the recipient, that is, “address” indicates the delivery destination (destination) of the package. The “recipient name” indicates the name of the recipient.

FIG. 4 is a diagram illustrating an example of a data structure of each of a package-delivery route DB 41, a delivery route DB 42, and a package group DB 43 stored in the memory 11 of the server 1.

The package-delivery route DB 41 is a database for associating a package group carried collectively by a delivery person on foot with a delivery route stored in the delivery route DB 42, and stores a “package group ID” and a “delivery routing ID” in association with each other. The “package group ID” is an identifier assigned to a group of packages collected in advance. The “delivery route ID” is an identifier identifying a delivery route assigned to a package group indicated by a “package group ID”.

The delivery route DB 42 is a database for storing delivery route information indicating a delivery route indicated by a “delivery routing ID” described in the package-delivery route DB 41, in which one record is assigned to each piece of delivery route information. More specifically, the delivery route DB 41 stores a “delivery route ID” and a “delivery route” in association with each other.

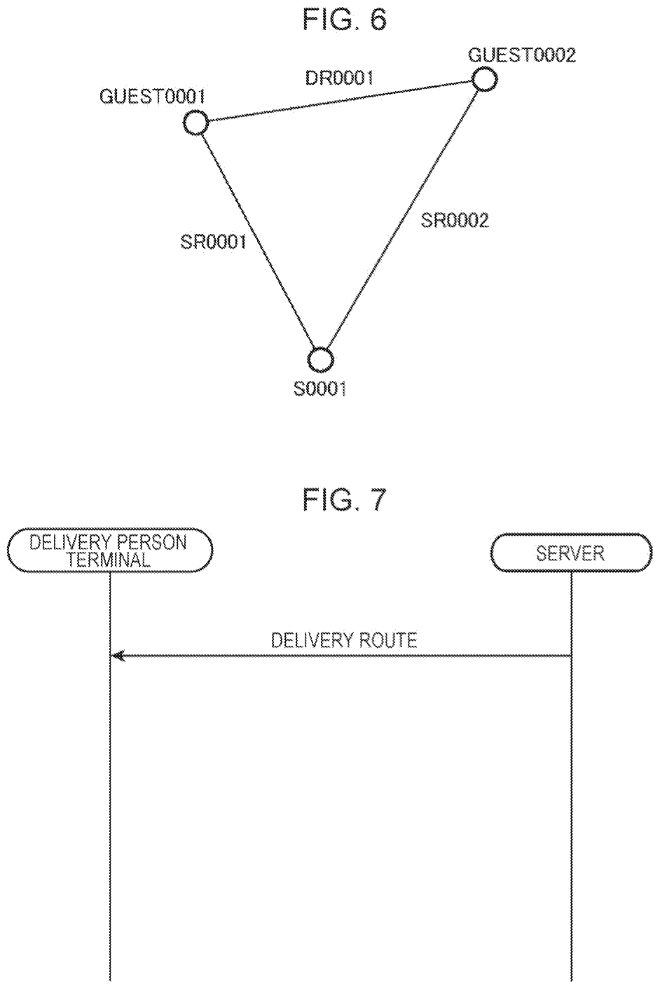

The “delivery route” indicates a content of a delivery route. The content of the “delivery route” will be described later with reference to FIG. 6.

The package group DB 43 is a database representing a content of a package group indicated by a “package group ID”, and stores a “package group ID” and “package IDs” in association with each other. The “package group ID” is an identifier assigned to the whole of a group of packages collected in advance. The “package ID” is a package ID of one of packages included in a group of packages indicated by a “package group ID”.

FIG. 5 is a diagram illustrating an example of a data structure of the route segment length DB 51 stored in the memory 11 of the server 1. The route segment length DB 51 is a database for storing information related to route segments connecting destinations on a delivery route indicated by delivery route information stored in the delivery route DB 42. The route segment length DB 51 stores a “destination #1” a “destination #2” a “route segment between destination #1 and destination #2”, and a “length” in association with each other. The “destination #1” indicates a destination upstream of a route segment. A user ID of a recipient of a package is stored in a field of the “destination #1”. The “destination #2” indicates a destination downstream of the route segment. A user ID of a recipient of a package is stored in a field of the “destination #2”. In a field of the “route segment between destination #1 and destination #2”, an identifier of a route segment connecting the destination indicated by the destination #1 and the destination indicated by the destination #2, such as “DR0001”, “DR0002”, or the like is described. The “distance” indicates the distance of a route segment. Note that the “distance” may be given by a linear distance between destinations, or a distance of an optimal route between destinations determined from a map image using a route search algorithm, that is, a distance of a route that a delivery person actually travels on foot. This applies also to the “distance” of the route segment length DB 52.

The route segment length DB 52 is a database for storing information related to a route segment connecting a delivery starting point and a destination of a delivery route segment on a delivery route indicated by delivery route information stored in the delivery route DB 42. In the route segment length DB 52, the “delivery starting point”, the “destination”, and the “route segment between delivery start point and destination”, and the “distance” are stored in association with each other. The “delivery start point” indicates a predetermined point where the delivery person stops a delivery vehicle to start to deliver a package on foot. The “destination” indicates a destination located next to the delivery start point. In the field of “destination”, a user ID of a recipient is stored. In a field of the “route segment between delivery start point and destination”, an identifier such as “SR0001”, “SR0002” or the like connecting the delivery start point and the destination such is stored. The “distance” indicates the distance of a route segment.

FIG. 6 is a diagram illustrating symbols used to indicate delivery routes stored in the delivery route DB 42. More specifically, in the example illustrated in FIG. 6, a delivery route indicated by a delivery route ID “TR0001” stored in a first row of the delivery route DB 42 is illustrated.

A symbol beginning with “S” such as “S0001” is an identifier of a delivery start point. Symbol beginning with “SR” such as “SR0001” are each an identifier of a route segment connecting a delivery start point and a destination. Symbol beginning with “GUEST” such as “GUEST0001” are each a recipient customer ID, that is, a destination.

Thus, the delivery route segment of “TR0001” starts from the delivery start point “S0001”, goes to the destination “GUEST0001” via the route segment “SR0001”, and further goes to a destination “GUEST 0002” via a route segment “DR0001”, and then returns to the delivery start point “S0001” via a route segment “SR0002”.

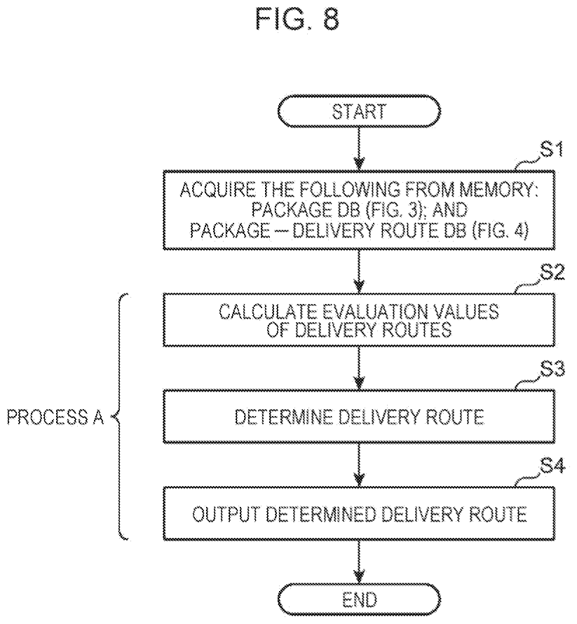

FIG. 7 is a sequence diagram illustrating an example of a sequence of data transmission/reception between the server 1 and the delivery person terminal 2 in the information providing system illustrated in FIG. 1. For example, when the delivery person stops the delivery vehicle at the delivery start point, the server 1 determines an optimum delivery route having a minimum evaluation value among delivery routes corresponding to the delivery start point, and transmits information indicating the determined delivery route to the delivery person terminal 2.

FIG. 8 is a flowchart illustrating an example of a process performed by the information providing system according to Embodiment 1. In S1, the control unit 15 of the server 1 acquires the package DB 31 and the package-delivery route DB 41 from the memory 11.

In S2, the evaluation value calculator 12 acquires, from the delivery route DB 42, delivery routes corresponding to a package group including packages to be delivered by the delivery person on foot, and calculates an evaluation value of each delivery route. For example, let it be assumed here that the package IDs of packages in the package group to be delivered are “1234-5678-90” and “1234-5678-89”. In this case, the package group DB 43 is referred to and a package group ID “P0001” is acquired. Next, the package-delivery route DB 41 is referred to, and delivery route IDs “TR0001” and “TR0002” corresponding to the package group ID “P0001” are acquired. Next, the delivery route DB 42 is referred to, and two delivery routes indicated by the delivery route IDs “TR0001” and “TR0002” are acquired as delivery routes for which evaluation values are to be calculated. An evaluation value is calculated for each of these two delivery routes. Although two delivery routes are acquired in the specific example described above, the present disclosure is not limited thereto, and three or more delivery routes may be acquired.

In S3, the delivery route determiner 14 determines a delivery route having the smallest evaluation value as the optimum delivery route among the delivery routes for which the evaluation values were calculated in S2.

In S4, the communicator 13 transmits information indicating the optimum delivery route to the delivery person terminal 2. In response, the delivery person terminal 2 displays an image illustrating the optimum delivery route thereby presenting the optimum delivery route to the delivery person.

Note that in the evaluation value calculation in S2 by the evaluation value calculator 12, packages indicated by the group package ID including more destinations closer to the delivery start point currently assigned to the delivery person than other package groups may be extracted from the package group DB 43, and packages included in the extracted package group may be employed as those to be delivered.

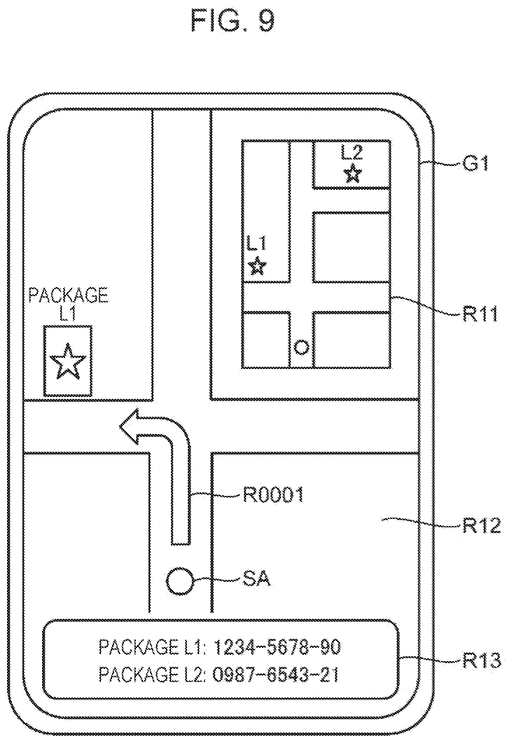

FIG. 9 is a diagram illustrating an example of an optimum delivery route displayed on a display screen G1 of the delivery person terminal 2. In this example, a delivery route is displayed along which the delivery person is to walk to deliver two packages L1 and L2 indicated by the package IDs “1234-5678-90” and “0987-6543-21”. In a display area R11, a map image including the delivery destination of the package L1 and the delivery destination of the package L2 is displayed to present an overall image of delivery route to the delivery person In a display area R12, a part of the map image displayed in the display area R11 is enlarged and displayed such that the it shows an enlarged delivery route R0001 from a delivery start point SA to a destination to which the package L1 is to be delivered by the delivery person. The map image displayed in the display area R12 may be an image obtained by enlarging the map image displayed in the display area R11 so as to include the current position and the destination according to the position information indicating the current position transmitted from the delivery person terminal 2.

In the display area R13, package IDs related to the respective packages that are to be collectively delivered by the delivery person on foot are displayed from the top to the bottom in the order of delivering. As described above, the destinations of the packages, the delivery order of the packages, the routes, and/or the like are displayed on the map image on the display screen G1, so as to properly guide the delivery person to deliver the packages along the optimum delivery route.

Thus, according to the present embodiment, the evaluation values are calculated for the respective delivery routes passing the destinations in order starting from the delivery start point and returning to the delivery start point based on not only the length information of each delivery route but also package information regarding the size or weight of each of the packages to be delivered by the delivery person on foot, and the optimum delivery route is determined based on the calculated evaluation values. Therefore, according to the present embodiment, it is possible to accurately calculate a low-load delivery route that allows the delivery person to efficiently deliver packages on foot from the delivery start point.

Furthermore, in the present embodiment, the optimum delivery route is determined based on the evaluation values calculated using the package information, and thus the determined optimum delivery route allows a reduction in the physical load on the delivery person.

The delivery route determined in the above-described manner is displayed on the delivery person terminal 2, which makes it possible to achieve high efficiency in delivering packages by the delivery person on foot. Therefore, even a new delivery person can deliver packages with high efficiency similar to that with which an experienced delivery person can.

### Embodiment 2

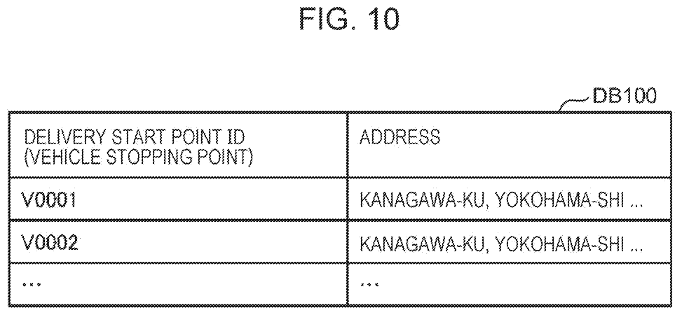

In Embodiment 2 described below, an optimum delivery route is determined using a delivery start point DB in which a predetermined delivery start point is stored. FIG. 10 is a diagram illustrating an example of a data structure of a delivery start point DB 100 according to Embodiment 2. The delivery start point DB 100 is a database for storing delivery start points in advance in the memory 11 of the server 1 in which one record is assigned to one delivery start point. In the present embodiment, the same components as those in Embodiment 1 are denoted by the same reference numerals, and a further description thereof is omitted. In the present embodiment, FIGS. 1 and 2 are used to explain a network configuration and a block configuration. These also apply to the following embodiments unless otherwise stated.

In the delivery start point DB 100, a “delivery start point ID” and an “address” are stored in association with each other. The “delivery start point ID” is an identifier of a delivery start point managed by the server 1. The “address” indicates an address of the delivery start point. As for the delivery start point stored in the delivery start point DB 100, a predetermined point where a delivery vehicle has been parked in the past is employed. For example, a place where the delivery vehicle can be easily stopped, such as a parking lot, is employed.

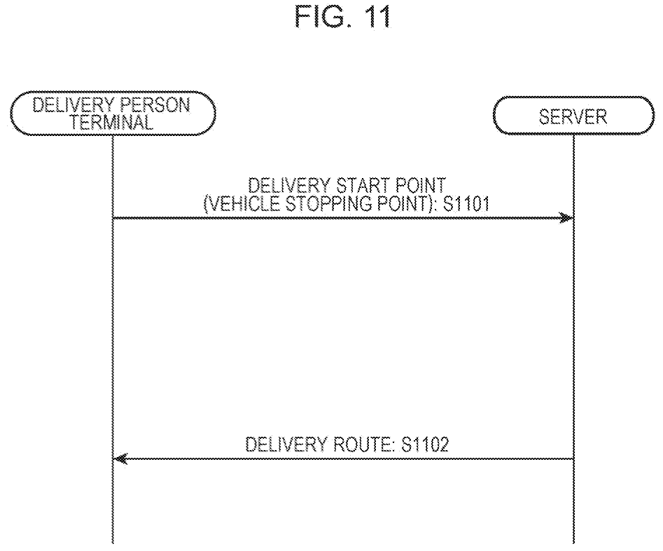

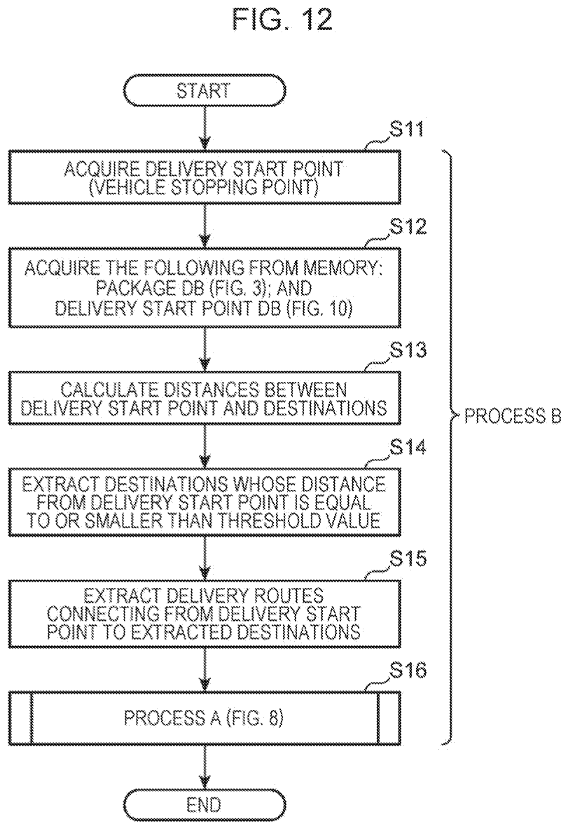

FIG. 11 is a sequence diagram illustrating an example of a sequence of data transmission/reception between the server 1 and the delivery person terminal 2 in the information providing system according to Embodiment 2. In the present embodiment, it is assumed that the delivery person knows which one of the delivery start points managed by the server 1 the delivery person is to go to. When the delivery vehicle arrives at the delivery start point, the delivery person terminal 2 transmits the delivery start point to the server 1 (S1101). More specifically, the delivery person terminal 2 may store the delivery start point in the memory 21 in advance. A current position detected by the GPS 22 is compared to the delivery start point stored in the memory 21. If the current position detected by the GPS 22 indicates that the current position is at the delivery start point, the delivery start point may be transmitted to the server 1.

Next, the server 1 determines the optimum delivery route based on the delivery start point transmitted from the delivery person terminal 2, and transmits the determined optimum delivery route to the delivery person terminal 2 (S1102).

FIG. 12 is a flowchart illustrating an example of a process performed by the information providing system according to Embodiment 2. In S11, using the communicator 13, the control unit 15 of the server 1 acquires the delivery start point transmitted from the delivery person terminal 2. In S12, the control unit 15 acquires the package DB 31 and the delivery start point DB 100 from the memory 11.

In S13, the control unit 15 calculates the distance between the delivery start point acquired in S11 and each of the destinations of the respective packages stored in the package DB 31. The destinations of the packages are detected by referring to the “address” of the customer DB 32 using, as a key, the “sender customer ID” of the package DB 31. The position information of the delivery start point and the destination may be given by the latitude and the longitude associated with the “address” in the map information. The distances between the delivery start point and the respective destinations may be calculated using the position information including the latitudes and the longitudes.

In S14, the control unit 15 extracts destinations for which the distance between the delivery start point and the destination calculated in S13 is equal to or less than a threshold value. As a result, among the packages stored in the package DB 31, packages are extracted whose destinations are located in a range of distance from the delivery start point smaller than a threshold value. As the threshold value, a distance value that is appropriate for the delivery person to carry a package on foot is selected in advance. For example, a distance of 10 m, 50 m, 100 m, or 500 m is employed.

In S15, the control unit 15 extracts delivery routes connecting the delivery start point and the destinations extracted in S14 starting from the delivery start point. More specifically, in this step, the control unit 15 may extract, from the package group DB 43, package IDs of packages whose destinations are extracted in S14, and may further extract, from the delivery route DB 42, delivery routes corresponding to the extracted package group ID thereby extracting delivery routes.

In S16, a process A is executed on the delivery routes extracted in S15. More specifically, in the process A, S2 to S4 illustrated in FIG. 8 are executed.

Thus, according to the present embodiment, evaluation values are calculated for the respective delivery routes including delivery destinations whose distances from the delivery start point are equal to or smaller than the threshold value, and an optimum delivery route is calculated using the calculated evaluation values. Thus, it is possible to prevent a delivery destination, whose distance from the delivery start point is larger than the threshold value and thus which is difficult to get to on foot, from being set as a destination.

### Embodiment 3

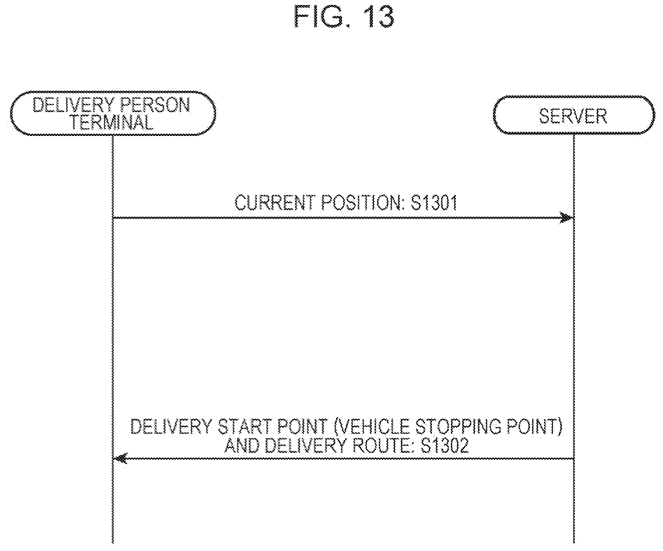

In Embodiment 3 described below, among candidates for the delivery start point stored in the delivery start point DB 100, a candidate for the delivery start point located closest to the current position of the delivery person is determined as the delivery start point. FIG. 13 is a sequence diagram illustrating an example of a sequence of data transmission/reception between the server 1 and the delivery person terminal 2 in the information providing system according to Embodiment 3.

In this embodiment, it is assumed that the delivery person does not know the delivery start points managed by the server 1. When the delivery person terminal 2 accepts an instruction input by the delivery person to transmit a notification of a current position, the delivery person terminal 2 transmits to the server 1 the current position detected by the GPS 22 (S1301). In response, the server 1 determines the delivery start point closest to the current position and also determines the optimum delivery route starting from the delivery start point. The server 1 transmits the determined delivery start point and optimum delivery route to the delivery person terminal 2 (S1302).

The delivery person terminal 2 displays, on the display unit 26, a display screen indicating the delivery start point and the optimum route received from the server 1. The delivery persons views this display screen and moves the delivery vehicle to the delivery start point indicated by the display screen, stops the delivery vehicle there, and delivers packages along the delivery route indicated by the display screen.

In the above-described process, the delivery person terminal 2 transmits the current position to the server 1 in response to the inputting, by the delivery person, of the instruction to transmit the current position, but this is merely an example, and the current position may be transmitted to the server 1 in response to detecting stopping of the delivery vehicle.

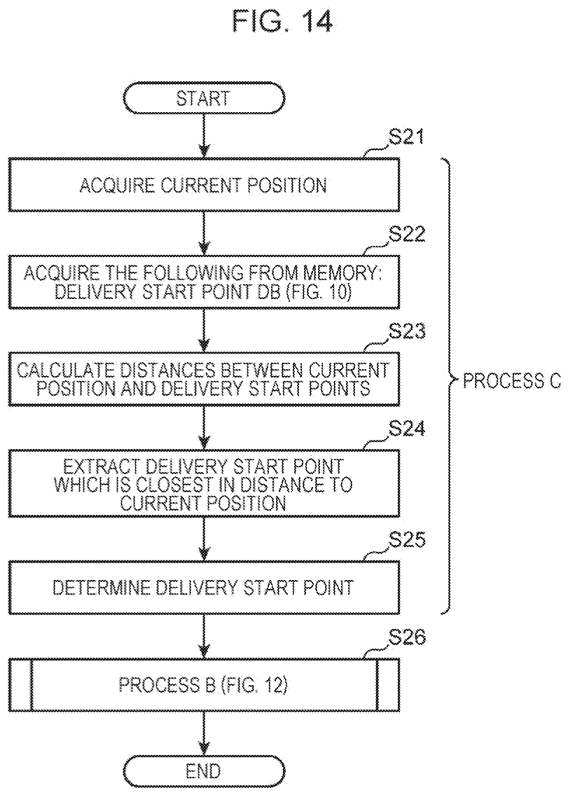

FIG. 14 is a flowchart illustrating an example of a process performed by the information providing system according to Embodiment 3. In S21, using the communicator 13, the control unit 15 of the server 1 acquires the current position transmitted from the delivery person terminal 2. In S22, the control unit 15 acquires the delivery start point DB 100 from the memory 11.

In S23, the control unit 15 calculates the distance between the current position and each of all the delivery start points stored in the delivery start point DB 100. In this step, for example, the control unit 15 may calculate a straight line distance between the current position and each delivery start point as the distance, or may calculate a distance along an optimum route between the current position and the delivery start point as the distance.

In S24, a delivery start point closest to the current position is extracted from the delivery start points whose distances are calculated in S23.

In S25, the control unit 15 determines the delivery start point extracted in S24 as the delivery start point to be notified to the delivery person.

In S26, a process B is executed using the delivery start point determined in S25. More specifically, in the process B, S11 to S16 illustrated in FIG. 12 are executed.

Thus, according to the present embodiment, since the delivery start point located closest to the current position of the delivery person is selected from the delivery start point DB 100 and notified to the delivery person, the delivery person can start delivering the packages on foot from an appropriate delivery start point near the current position even when the delivery person does not know the delivery start point.

### Embodiment 4

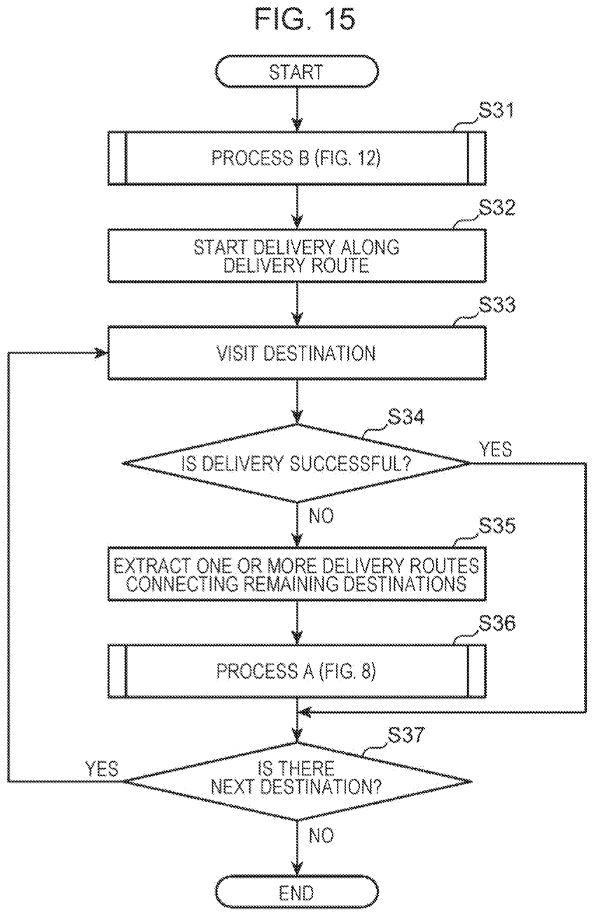

In Embodiment 4 described below, when a delivery person visits a destination, if a recipient is absent at the destination, a delivery route is rescheduled. FIG. 15 is a flowchart illustrating an example of a process performed by the information providing system according to Embodiment 5.

In S31, the process B is executed. More specifically, in the process B, S11 to S16 illustrated in FIG. 12 are executed. That is, in S31, the optimum delivery route passing through destination starting from the delivery start point and returning to the delivery start point is determined.

In S32, the control unit 15 detects that the delivery person has started delivery along the delivery route determined in S31. In S33, the control unit 15 detects that the delivery person has visited the destination. More specifically, for example, the control unit 15 may monitor position information periodically transmitted from the delivery person terminal 2 to detect starting delivering and visiting a destination by the delivery person.

In S34, the control unit 15 determines whether the delivery person has delivered the package successfully. In a case where the delivery person completes the delivery of the package to the recipient, the control unit 15 controls the reader 24 to read a bar code or a QR code (registered trademark) described on a package slip thereby acquiring a package ID, and the control unit 15 further controls the delivery person terminal 2 to transmit, to the server 1, information indicating that the package delivery is completed. Thus, when this information is received by the communicator 13, the control unit 15 may determine that the package has been delivered successfully.

In a case where the package is delivered successfully in S34 (YES in S34), the process proceeds to S37. However, in a case where the delivering of the package is not successful in S34 (NO in S34), the process proceeds to S35.

In S35, the control unit 15 extracts one or more delivery routes by using the delivery start point and the remaining one or more destinations on the delivery routes determined in S31 to which delivering has not yet been completed. Note that in this step, the control unit 15 extracts all combinations of delivery routes that pass through all remaining destinations and return to the delivery start point. For example, in a case where there are destinations M1 and M2 as the remaining destinations, and the current position is M0 and the delivery start point is S0, then two delivery routes, that is, a delivery route M0-M1-M2-S0 and a delivery route M0-M2-M1-S0 are extracted. Here, a destination located at the current position M0 corresponds to an example of the first destination, and a recipient at this destination corresponds to an example of the first recipient. The delivery routes extracted in S35 are examples of the second delivery routes.

In S36, the process A is executed on the delivery routes extracted in S35. That is, the evaluation values of the respective delivery routes are calculated, and a delivery route having the smallest evaluation value is determined as the optimum delivery route. Note that, in the process A, S2 to S4 illustrated in FIG. 8 are executed.

In S37, the control unit 15 determines whether or not there is a next destination. For example, when at the current position M0, the route M0-M1-M2-S0 described above is determined as the optimum delivery route, if the delivery of packages to the destinations of M1 and M2 is not yet completed, the determination result in S37 is YES, and the process returns to S33. When the delivery route M0-M1-M2-S0 is employed, if the delivery to M2 is completed, the determination result in S37 is NO, and thus the process is ended.

In a case where a recipient of the package is absent at a destination, the delivery person has to carry this package as an extra package and deliver the remaining packages to the remaining destinations, which causes a possibility that the initially calculated delivery route is not proper.

Therefore, in the present embodiment, when a package recipient is absent at a certain destination, the delivery route connecting the remaining destinations in order is rescheduled. That is, an optimum delivery route is determined taking into account the weights or sizes of undelivered packages.

### Embodiment 5

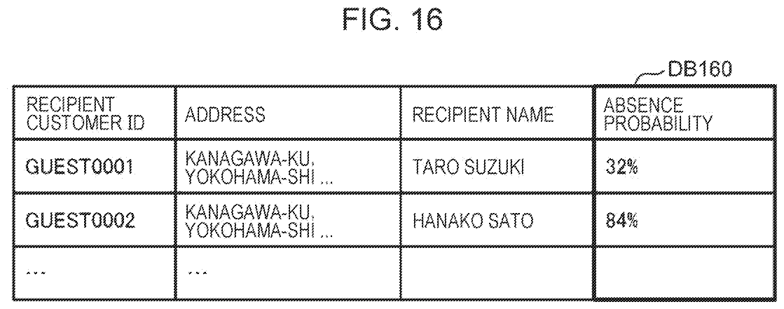

In Embodiment 5 described below, evaluation values are calculated using history information indicating ratios of redelivery for destinations made in the past. FIG. 16 is a diagram illustrating an example of a data structure of a customer DB 160 stored in the memory 11 of the server 1 according to Embodiment 5. The customer DB 160 further includes a field of “absence probability” in addition to the fields included in the customer DB 32 illustrated in FIG. 3. The “absence probability” indicates the probability that the recipient was absent when a package was tried to be delivered in the past, that is, the “absence probability” is the redelivery ratio in the past. The memory 11 stores a delivery log (not shown) in which the date and time of delivering a package and information indicating whether or not the package was actually delivered are recorded for each recipient (customer). The delivery log is recorded by the server 1 when the server 1 acquires a delivery result input to the delivery person terminal 2 by the delivery person each time the delivery person visits a destination. Thus, the “absence probability” can be obtained by calculating the ratio of the number of absence to the total number of deliveries stored in the delivery log for each customer. The customer DB 160 is an example of history information.

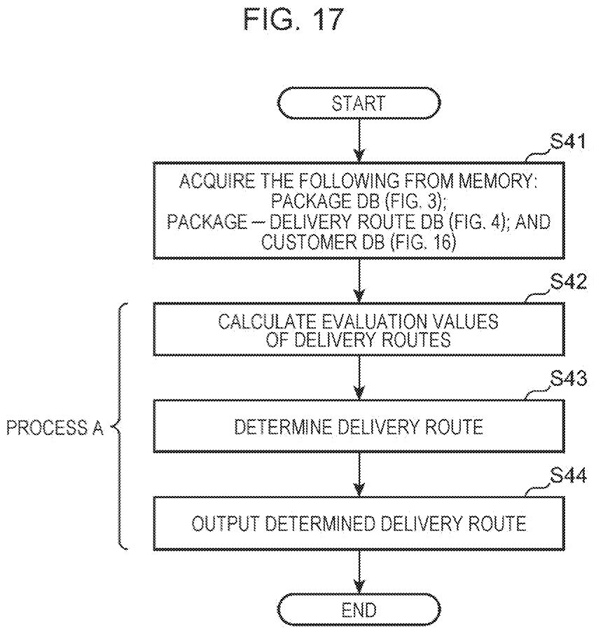

FIG. 17 is a flowchart illustrating an example of a process performed by the information providing system according to Embodiment 5. In S41, the control unit 15 acquires the package DB 31, the package-delivery route DB 41, and the customer DB 160 from the memory 11.

In S42 to S44, the process A is executed further using the “absence probability” stored in the customer DB 160, and thus the optimum delivery route is calculated. More specifically, in the process A, S2 to S4 illustrated in FIG. 8 are executed. The evaluation value taking into account the absence probability will be described later with reference to a specific example of a delivery route.

According to the present embodiment, as described above, the evaluation values are calculated taking into account the absence probability, which makes it possible to determine more realistic the evaluation values. Thus, it is possible to achieve enhanced reliability in the finally determined delivery route.

### Embodiment 6

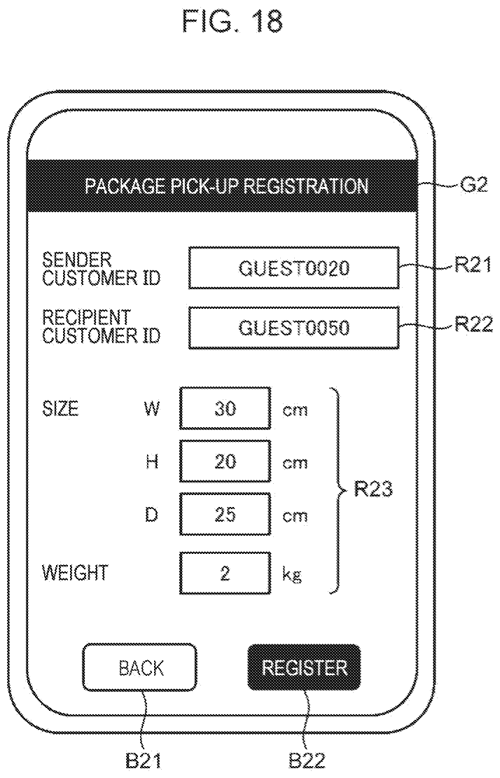

In Embodiment 6 described below, when a package to be delivered is picked up at a destination, the delivery route is rescheduled immediately at that destination. FIG. 18 is a diagram illustrating an example of a display screen G2 displayed on a delivery person terminal according to Embodiment 6. The display screen G2 is a screen for notifying the server 1 of having picked up a package to be delivered at a destination. Picking-up means to pick up a package from a customer at a destination when the delivery person visits the destination.

The display screen G2 includes a package sender ID inputting area R21, a package recipient ID inputting area R22, an inputting area R23 for inputting a size and a weight of a picked-up package, a back button B21, and a registration button B22. In the inputting area R21, a customer ID indicating a sender of a picked-up package is input by a delivery person. In the inputting area R22, a customer ID indicating a recipient of the picked-up package is input by delivery person. In the inputting area R23, a width (W), a height (H), a depth (D), and a weight of the picked-up package are input by the delivery person.

The back button B21 is a button for returning the display screen G2 back to a previously displayed display screen. In a case where the delivery person picks up a package at a destination, the delivery person inputs necessary information in each input area of the display screen G2 and presses the registration button B22. In response, the communicator 25 of the delivery person terminal 2 transmits the information input in each inputting area to the server 1.

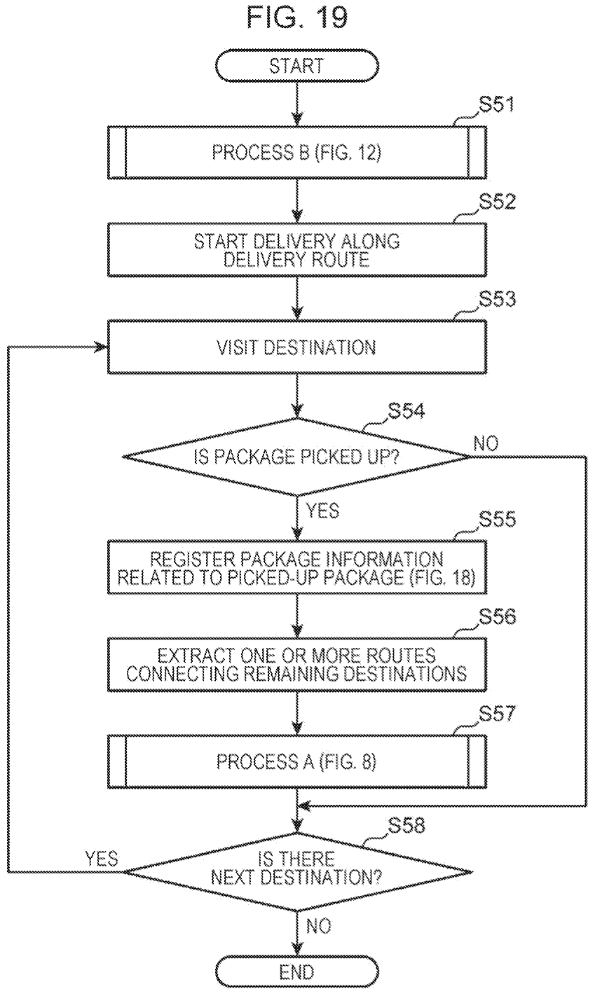

FIG. 19 is a flowchart illustrating an example of a process performed by the information providing system according to Embodiment 6. S51 to S53 are the same as S31 to S33 in FIG. 15. In S54 following S53, the control unit 15 determines whether or not a package was picked up. More specifically, in this step, in a case where the control unit 15 receives from the delivery person terminal 2 information indicating that a package has been picked up in addition to information indicating that a package has been delivered to a recipient at a destination, the control unit 15 may determine that the package has been picked up.

In a case where a package is not picked up (NO in S54), the process proceeds to S58, but in a case where a package is picked up (YES in S54), the process proceeds to S55.

In S55, the control unit 15 acquires the package information regarding the picked-up package (an example of the second package information) from the delivery person terminal 2 using the communicator 13, and stores the acquired package information in the memory 11. More specifically, the control unit 15 may acquire the information input via the display screen G2 illustrated in FIG. 18 as the package information on the picked-up package and may store it in the memory 11.

In S56, one or more delivery routes connecting the remaining destinations are extracted. The details of this process are the same as the process in S35 in FIG. 15.

In S57, the process A is performed on the delivery routes extracted in S56 while applying the package information acquired in S55, thereby calculating the evaluation values of the respective delivery routes and determining the optimum delivery route having the minimum evaluation value. S58 is the same as S37 in FIG. 15.

When a package is picked up at a destination on the way, the addition of this package may make it difficult to efficiently deliver packages using the original delivery route.

In the present embodiment, to handle the above situation, when a package is picked up at a certain destination, a delivery route connecting this destination to the remaining destinations is rescheduled taking into account the size or weight of the picked-up package. That is, an optimum delivery route is determined taking into account the weight or size of the picked-up package.

The picked-up package is carried to the delivery start point, loaded on a delivery vehicle, transported to a delivery center, and delivered to a corresponding destination by another delivery vehicle.

### Embodiment 7

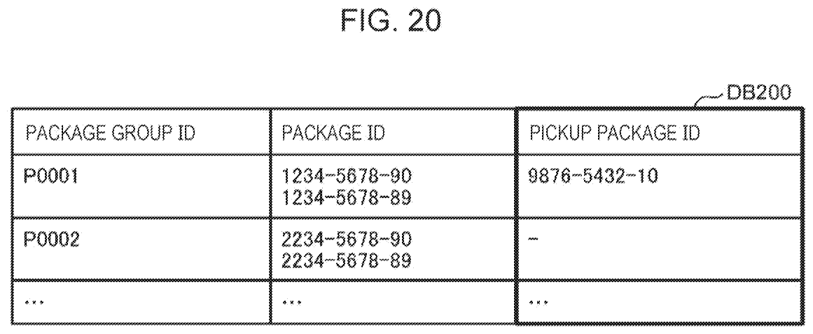

In Embodiment 7 described below, an optimum delivery route is determined in a situation in which package information on a package to be picked up at a destination is stored in advance in the memory 11, FIG. 20 is a diagram illustrating an example of a data structure of a package group DB 200 stored in the memory 11 of the server 1 according to Embodiment 7.

The package group DB 200 further includes a field of “pickup package ID” in addition to the fields of the package group DB 43. The “pickup package ID” indicates a package ID of a package to be picked up at a certain destination. In a first row of the package group DB 200, two packages described in a field of “package ID” and one package described in the filed “pickup package ID” are combined together and a “pickup package ID” is given thereto.

Note that the package information related to the package to be picked up is stored in advance in the package DB 31 illustrated in FIG. 3, and the destination where the package is to be picked up and the delivery destination of the picked-up package are determined from this package information.

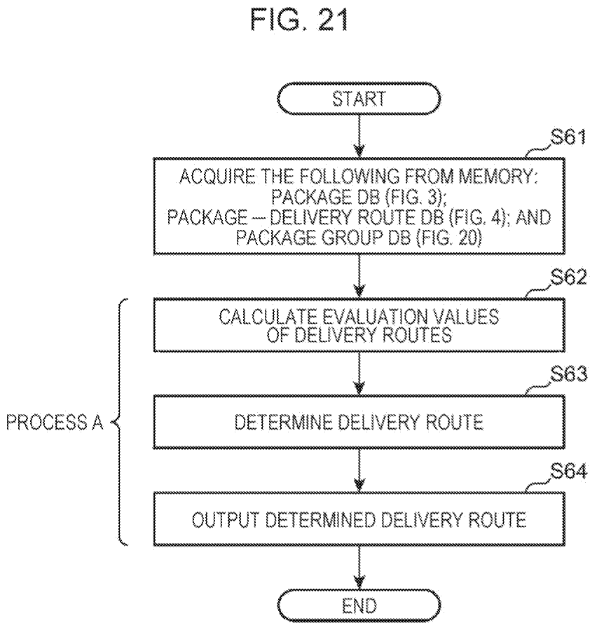

FIG. 21 is a flowchart illustrating an example of a process performed by the information providing system according to Embodiment 7. In S61, the control unit 15 acquires the package DB 31, the package-delivery route DB 41, and the package group DB 43 from the memory 11.

The process A is executed in S62 to S64. Note that in S62, the evaluation value of the respective delivery routes are calculated taking into account the weights or sizes of the packages to be picked up in addition to the packages to be delivered.

According to the present embodiment, as described above, when the sizes or weights of packages to be picked up are known in advance, the optimum delivery route is determined taking into account not only the packages to be delivered but also the packages to be picked up. As a result, a highly efficient delivery route can be presented to the delivery person without rescheduling.

### Embodiment 8

In Embodiment 8 described below, when the delivery vehicle cannot be stopped at a delivery start point, a delivery start point next closest to the current position is determined.

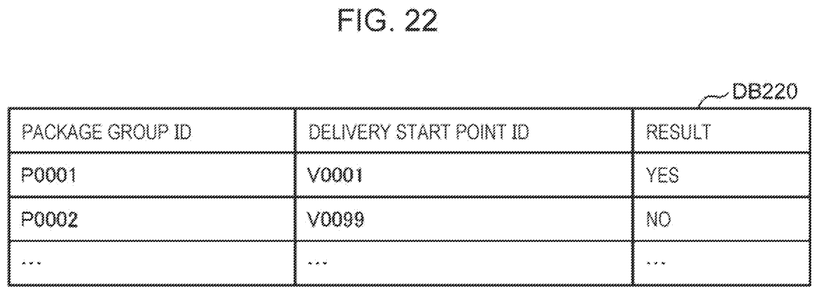

FIG. 22 is a diagram illustrating an example of a data structure of a package group-delivery start point DB 220 stored in the memory 11 of the server 1 according to Embodiment 8. The package group-delivery start point DB 220 is a database that stores information indicating whether or not the delivery vehicle could be stopped at the delivery start point. In this database, the “package group ID”, the “delivery start point ID”, and the “result” are stored in association with each other.

The “package group ID” is the same as that described in the package group DB 43 illustrated in FIG. 4. The “delivery start point ID” is an identifier of the delivery start point from which delivering of packages specified by the “package group ID” is to be started. In the field of “result”, a result indicating whether or not the delivery vehicle could be stopped at a delivery start point is described.

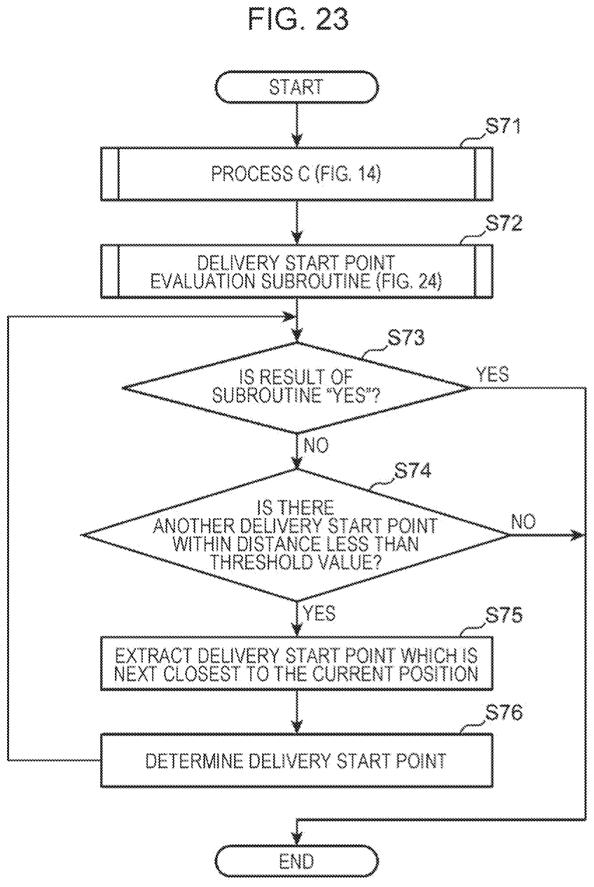

FIG. 23 is a flowchart illustrating an example of a process performed by the information providing system according to Embodiment 8. In S71, a process C is executed. More specifically, in the process C, S21 to S25 illustrated in FIG. 14 are executed.

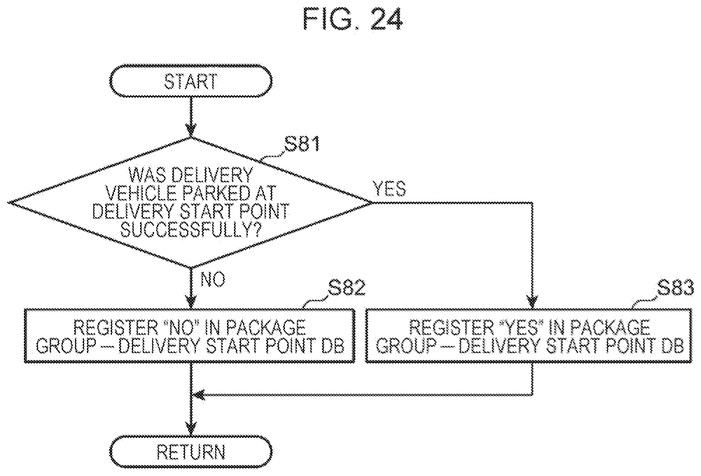

In S72, a delivery start point evaluation routine is executed. Details of this processing will be described later with reference to FIG. 24.

In S73, the control unit 15 determines whether or not the result of the routine in S72 is YES. In a case where the result of the routine in S72 is YES (YES in S73), the process is ended, while in a case where the result of the routine in S72 is NO (NO in S73), the process proceeds to S74. That is, in a case where the delivery vehicle cannot be stopped at the delivery start point determined in the process C, it is necessary to determine a next delivery start point, and thus the process proceeds to S74. The determination of whether the result of the routine is YES or NO is made based on the information stored in the “result” field of the package group-delivery start point DB 220.

In S74, the control unit 15 refers to the delivery start point DB 100 and determines whether or not there is another delivery start point within a distance, from the current position, equal to or less than a threshold value.

In a case where there is another delivery start point (YES in S74), the process proceeds to S75. However, in a case where there is not another delivery start point (NO in S74), the process is ended. Here, the current position is, for example, a position where the delivery person stops the delivery vehicle in the vicinity of the original delivery start point after the delivery person unsuccessfully tries to stop the delivery vehicle at the original delivery start point.

In S75, the control unit 15 extracts, from the delivery start point DB 100, a delivery start point whose distance from the current position is the next closest to the original delivery start point.

In S76, the control unit 15 determines the delivery start point extracted in S75 as a new delivery start point, and advances the process to S73.

FIG. 24 is a flowchart illustrating details of the process of the delivery start point evaluation subroutine in S72 illustrated in FIG. 23. In S81, the control unit 15 determines whether or not the delivery vehicle was parked successfully at the delivery start point. More specifically, for example, when the position information periodically transmitted from the delivery person terminal 2 indicates that the delivery vehicle has stopped at a location near the delivery start point for a particular time or more, the control unit 15 may determine that the delivery vehicle could not be stopped at the delivery start point. On the other hand, in a case where the position information periodically transmitted from the delivery person terminal 2 indicates that the delivery vehicle has stopped at the delivery start point for a particular time or more, the control unit 15 may determine that the delivery vehicle could be stopped at the delivery start point.

In a case where it is determined in S81 that the delivery vehicle can be parked at the delivery start point (YES in S81), the control unit 15 performs control such that YES is registered in the “result” field in a corresponding record of the delivery start point in the package group-delivery start point DB 220 (S83). In a case where it is determined that the delivery vehicle cannot be parked at the delivery start point (NO in S81), the control unit 15 performs control such that NO is registered in the “result” field in the corresponding record of the delivery start point in the package group-delivery start point DB 220. When the process in S82 and S83 is completed, the process returns to FIG. 23.

According to the present embodiment, as described above, in the case where the delivery vehicle cannot be stopped at the original delivery start point, a delivery start point whose distance from the current position is the second closest after the original delivery start point is employed, and thus the delivery person is allowed to stop the delivery vehicle at the easy-to-stop delivery start point which allows it to efficiently delivery packages.

### Embodiment 9

In Embodiment 9, in a case where the delivery vehicle cannot be stopped at the delivery start point, the delivery person inputs a reason why the delivery vehicle cannot not be stopped.

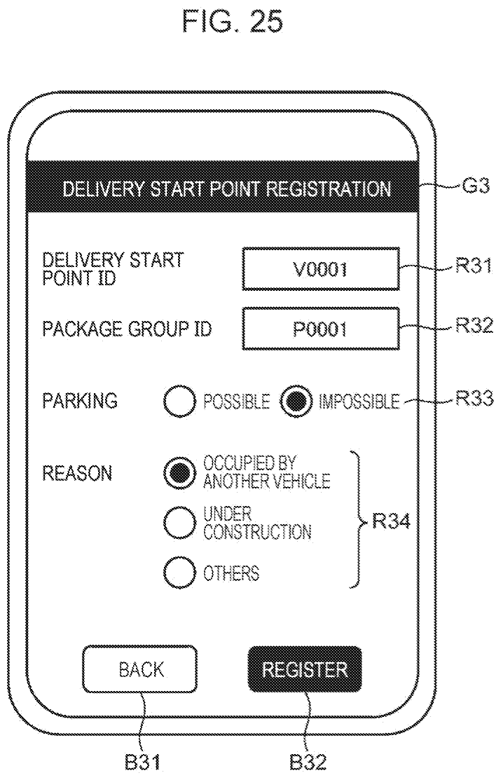

FIG. 25 is a diagram illustrating an example of a reason registration screen G3 displayed on the delivery person terminal 2 according to Embodiment 9. A reason registration screen G3 is a screen displayed on the delivery person terminal 2 when the delivery vehicle cannot be stopped at the delivery start point.

The reason registration screen G3 includes a display area R31 for displaying a delivery start point ID, a display area R32 for displaying a package group ID, an inputting area R33 for inputting a stop result, a reason inputting area R34, a back button B31, and a registration button B32.

In the display area R31, a delivery start point ID of a delivery start point where the delivery vehicle could not be stopped is displayed. More specifically, the server 1 may identify the delivery start point ID of the delivery start point where the delivery vehicle could not be stopped and may display it in advance in the display area R31. In the display area R32, a package group ID is displayed. More specifically, the server 1 may identify the package group ID corresponding to the delivery start point where the delivery vehicle could not be stopped and may display the package group ID in advance in the display area R32. In the input area R33, when the delivery vehicle could be stopped, a button “possible” is selected by the delivery person, while when the delivery vehicle could not be stopped, a button “impossible” is selected by the delivery person.

The inputting area R34 is an area in which the reason why the delivery vehicle cannot be stopped is input by the delivery person. In the present example, the delivery person is allowed to select one of the three options “occupied by parking vehicle”, “under construction”, and “others”. The option of “occupied by parking vehicle” is selected when the delivery start point is occupied by another parking vehicle. The option “under construction” is selected when the delivery start point is under construction. The option “others” is selected when the delivery vehicle cannot be stopped for a reason other than the above-described two reasons.

The back button B31 is a button that is pressed to return back to a previous screen displayed before the reason registration screen G3 is displayed. When the delivery person cannot stop the delivery vehicle at the delivery start point, he/she inputs necessary information into each input area of the reason registration screen G3 and presses the registration button B32. In response, the communicator 25 of the delivery person terminal 2 transmits the information input in each inputting area to the server 1.

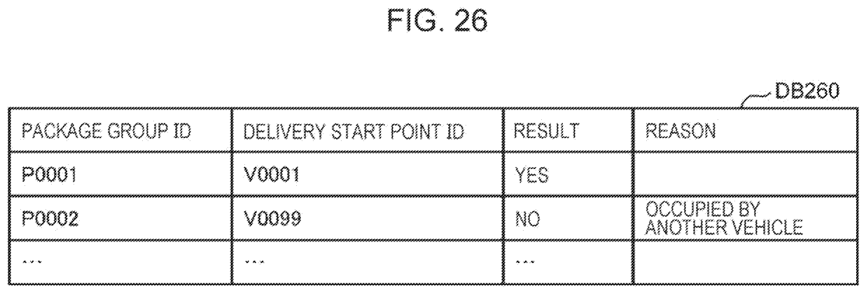

FIG. 26 is a diagram illustrating an example of a data structure of a package group-delivery start point DB 260 stored in the memory 11 of the server 1 according to Embodiment 9. The package group-delivery start point DB 260 further includes a field of “reason” in addition to the fields of the package group-delivery start point DB 220 illustrated in FIG. 22. The “reason” indicates the reason why the delivery vehicle input by the delivery person using the reason registration screen G3 could not be stopped.

In the specific example illustrated in FIG. 26, the delivery vehicle could be stopped at a delivery start point in a first row, and thus the field of “reason” in the first row is blank. At a delivery start point in a second row, the delivery vehicle could not be stopped because the delivery start point was occupied by another parking vehicle, and thus “occupied by another vehicle” is input in the field of “reason” in the second row.

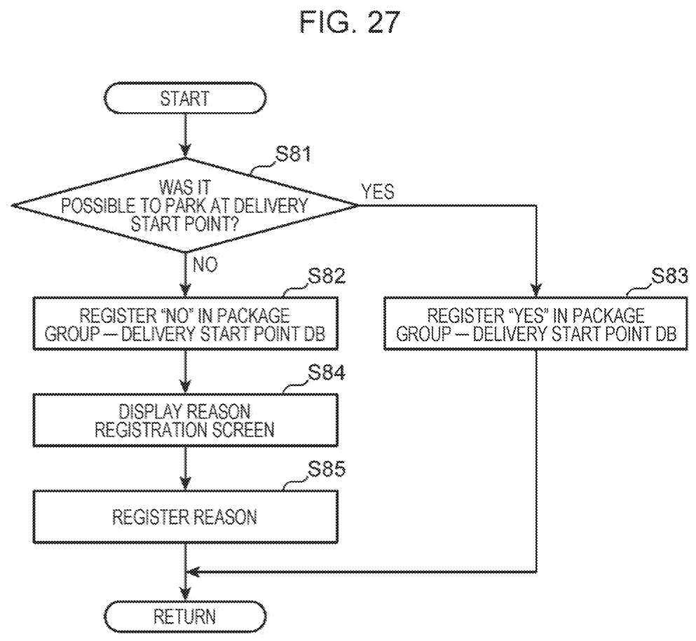

FIG. 27 is a flowchart illustrating an example a process of a delivery start point evaluation subroutine according to Embodiment 9. Note that the main routine in the present embodiment is the same as that illustrated in FIG. 23. In the flow chart in FIG. 27, the same steps as those in FIG. 24 are denoted by the same step numbers.

In S84, the control unit 15 performs control such that the reason registration screen G3 is displayed on the display unit 26 of the delivery person terminal 2. In S85, under the control of the control unit 15, the reason why the delivery vehicle could not stopped, selected by the delivery person via the reason registration screen G3, is registered in the field of the “reason” of the package group-delivery start point DB 260. When the process in S83 or S85 is completed, the process returns to FIG. 23.

In the present embodiment, as described above, when the delivery vehicle cannot be stopped, the reason therefor is notified from the delivery person terminal 2 and is registered in the package group-delivery start point DB 260. Thus, it is possible to collect information based on which a determination is to be made as whether or not the points are proper as delivery start points.

### Embodiment 10

In Embodiment 10, when the delivery vehicle can be stopped at a delivery start point, the delivery person inputs information indicating whether or not the delivery start point is preferable as a place where the delivery vehicle is to be stopped.

FIG. 28 is a diagram illustrating an example of an evaluation registration screen G4 displayed on the delivery person terminal 2 according to Embodiment 10. The evaluation registration screen G4 is a screen for use by the delivery person to input an evaluation on a delivery start point when the delivery vehicle can be stopped there.

The evaluation registration screen G4 is similar to the reason registration screen illustrated in FIG. 25 except that an evaluation inputting area R41 is provided instead of the reason inputting area R34. Other than that, the evaluation registration screen G4 is the same as the reason registration screen G3.

In the inputting area R41, the evaluation regarding the ease of parking at a delivery start point is input by the delivery person. Here, the inputting area R41 includes selection buttons for selecting an evaluation in evaluation items of “Is it easy to park?”, and “Is it easy to walk to destination?”. When it is easy to park, the delivery person selects a button “easy” for the evaluation item “Is it easy to park?”, while when it is difficult to park, the delivery person selects a button of “difficult”.

If the delivery route to a destination is easy to walk, the delivery person selects a button “easy” is selected for the evaluation item “Is it easy to walk to destination?”, while when it is difficult to walk, the delivery person selects a button “difficult”.

The inputting area R41 further includes a comment inputting area. In the comment inputting area, the delivery person inputs a comment regarding the delivery start point.

For example, the delivery person inputs a comment “there is a slope to the XX apartment” or the like. That is, in the comment inputting area, an arbitrary message may be input by the delivery person.

When the delivery vehicle could be stopped at a delivery start point, the delivery person inputs necessary information into each input area of the evaluation registration screen G4 and presses the registration button B32. In response, the communicator 25 of the delivery person terminal 2 transmits the information input in each inputting area to the server 1.

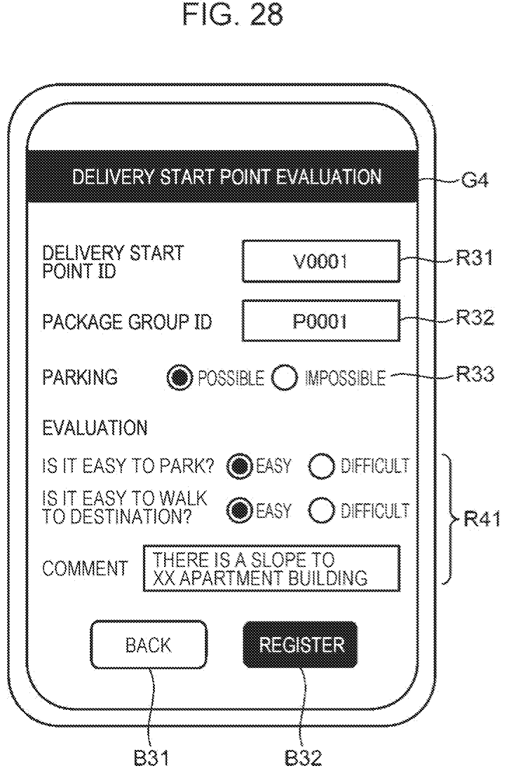

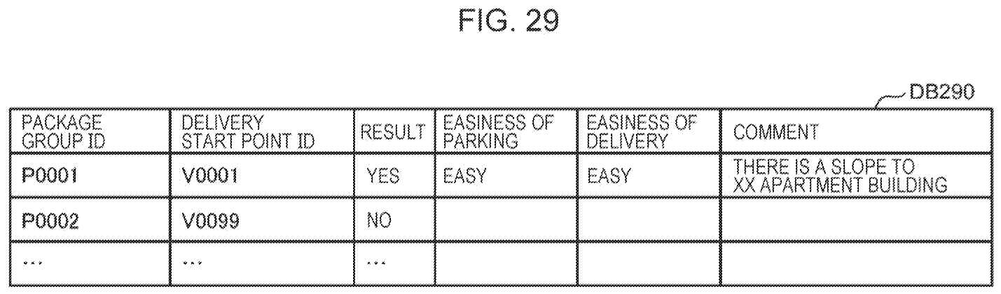

FIG. 29 is a diagram illustrating an example of a data structure of a package group-delivery start point DB 290 stored in the memory 11 of the server 1 according to Embodiment 10. The package group-delivery start point DB 290 further includes fields of “easiness of parking”, “easiness of delivery”, and “comment” in addition to the fields of the package group-delivery start point DB 260 illustrated in FIG. 26. In the fields of “easiness of parking”, “easiness of delivery”, and “comment”, the evaluation results made by the delivery person in terms of “easiness of parking”, “easiness of delivery”, and “comment” input via the evaluation registration screen G4 are stored.

For example, in a first row, the delivery vehicle could be stopped at a delivery start point, and thus evaluation results are described by the delivery person in the fields if “easiness of parking”, “easiness of delivery”, and “comments”. In a second row, the delivery vehicle could not be stopped at a delivery start point, and thus the fields of “easiness of parking”, “easiness of delivery”, and “comments” are blank.

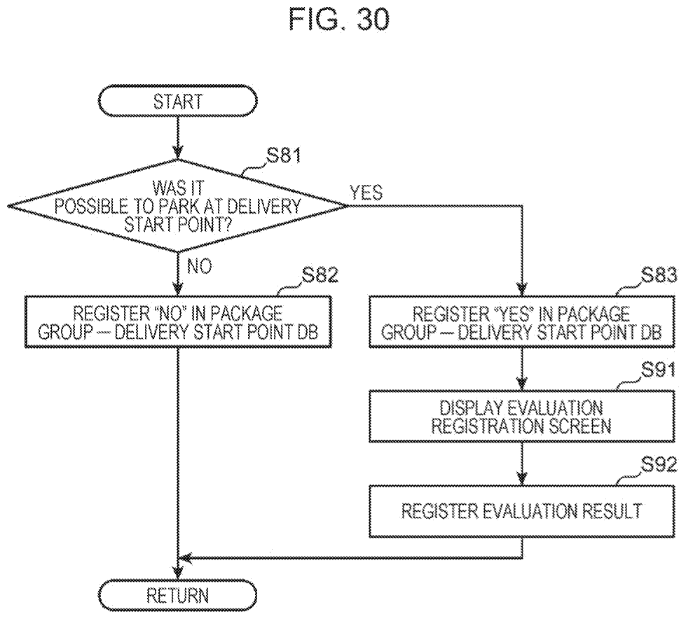

FIG. 30 is a flowchart illustrating an example a process of a delivery start point evaluation subroutine according to Embodiment 10. Note that the main routine in the present embodiment is the same as that illustrated in FIG. 23. In the flowchart in FIG. 30, the same steps as those in FIG. 24 are denoted by the same step numbers.

In S91, the control unit 15 performs control such that the evaluation registration screen G4 is displayed on the display unit 26 of the delivery person terminal 2. In S92, under the control of the control unit 15, the reason why the evaluation result could not stopped, selected by the delivery person via the evaluation registration screen G4, is registered in the field of the “reason” of the package group-delivery start point DB 290. When the process in S82 or S92 is completed, the process returns to FIG. 23.

According to the present embodiment, as described above, when the delivery vehicle can be stopped, the easiness of parking and the easiness of delivery at the delivery start point are notified. Thus, it is possible to collect information for determining whether the point is a proper delivery start point.

### Embodiment 11

In Embodiment 11, the total walkable distance on the delivery route is changed depending on the weather. FIG. 31 is a diagram illustrating an example of a network configuration of an information providing system according to Embodiment 11. The total walkable distance is the upper limit of the distance of the delivery route.

In the present embodiment, the system further includes a weather information providing server 3. The weather information providing server 3 is connected to the server 1 and the delivery person terminal 2 via a network NT such that they can communicate with each other.

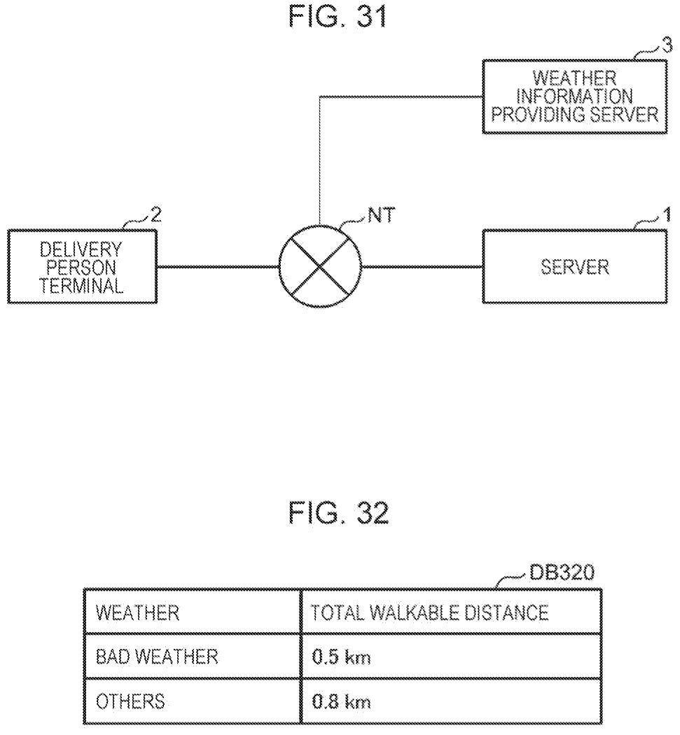

The weather information providing server 3 is a server that provides weather information including weather forecasts indicating, for example, fine weather, cloudy weather, or the like. FIG. 32 is a diagram illustrating an example of a data structure of a weather distance DB 320 stored in the memory 11 of the server 1 according to Embodiment 11.

A weather distance DB 320 is a database that stores the total walkable distances, which are upper limit distances on the delivery route for various weather conditions. In this weather distance DB 320, “weather” and “total walkable distance” are stored in association with each other. In the example illustrated in FIG. 32, “0.5 km” is stored as the “total walkable distance” for “bad weather”, and “0.8 km” is stored as the “total walkable distance” for “others”. Note that in FIG. 32, the “walkable total distance” for “bad weather” is an example of the second upper limit distance, and the “total walkable distance” for others is an example of the first upper limit distance. As described above, in the weather distance DB 320, the total walkable distance for bad weather is set to be shorter than in other cases.

Examples of “bad weather” includes rain, strong wind, snow, and the like, and “others” are, for example, fine weather and cloudy weather.

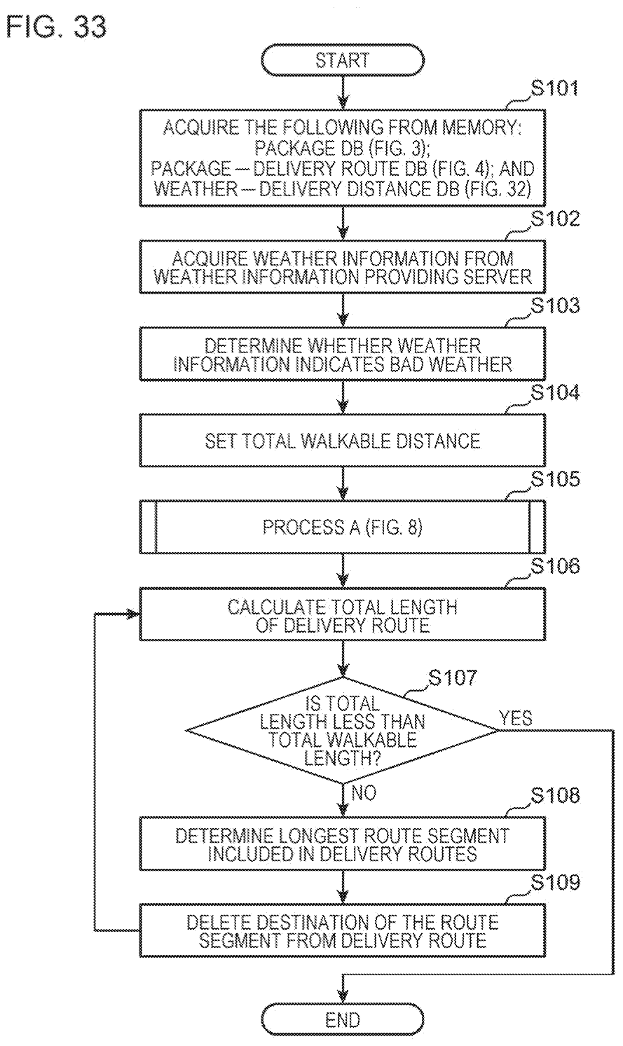

FIG. 33 is a flowchart illustrating an example of a process performed by an information providing system according to Embodiment 11. In S101, the control unit 15 acquires the package DB 31, the package-delivery route DB 41, and the weather distance DB 320 from the memory 11.

In S102, using the communicator 13, the control unit 15 of the server 1 acquires the weather information from the weather information providing server 3. Here, the weather information transmitted in this step is current weather information. In S103, the control unit 15 determines whether or not the weather is bad based on the weather information acquired in S102.

In S104, the control unit 15 refers to the weather distance DB 320 and sets the total walkable distance according to the determination result in S103. Here, when the determination result in S103 indicates a bad weather, 0.5 km is set as the total walkable distance, while when the determination result in S103 indicates a not bad weather, the total walkable distance is set to 0.8 km.

In S105, the process A is executed. As a result, a delivery route is determined. Note that, in the process A, S2 to S4 illustrated in FIG. 8 are executed. In S106, the control unit 15 calculates the total distance of the delivery route determined in S105. Here, the control unit 15 identifies the length of each of route segments included in the delivery route identified in S105 by referring to the route segment length DB 51 and the route segment length DB 52, and obtains the total length of each identified route thereby determining the total length the delivery route. For example, in the example of the delivery route in FIG. 6, the distances of the route segments “SR0001”, “DR0001”, and “SR0002” included in this delivery route are acquired from the route segment length DB 51 and the route segment length DB 52, and the total distance is calculated from the acquired values.

In S107, the control unit 15 determines whether or not the total distance calculated in S106 is smaller than the total walkable distance set in S104. In a case where the total distance is smaller than the walkable distance (YES in S107), the delivery route determined in S105 has a proper total distance for the current weather, and thus this delivery route is employed as the optimum delivery route, and the process is ended.

However, in a case where the total distance is equal to or greater than the walkable distance (NO in S107), the process proceeds to S108. In S108, the control unit 15 identifies a route segment with the longest distance among the route segments forming the delivery route determined in S105.

In the example of the delivery route described above, the route is composed of the three route segments “SR0001”, “DR0001”, and “SR0002”, and thus a route segment with the longest distance is identified from these route segments.

In S109, the control unit 15 deletes one of destinations located at upstream and downstream nodes of the longest route segment identified in S108 from the delivery route determined in S105. In a case where the longest route segment is a route segment connecting from the delivery start point to a destination, the control unit 15 may delete this destination. In a case where the longest route segment is a route segment connecting two destinations, the control unit 15 may delete a destination located at a downstream node. In a case where the longest route segment is a route segment connecting from a destination to the delivery start point, the control unit 15 may delete this destination.

When the process in S109 is completed, the process returns to S106. In S106, the total distance is calculated for the delivery route obtained as a result of deleting the destination in S109. In the above example of the delivery route, when the route segment “DR0001” is identified as the longest route segment, the destination “GUEST0002” located at the downstream node of the route segment “DR0001” is deleted, the total distance is calculated for the resultant delivery route. That is, in the processing flow illustrated in FIG. 33, deleting of a destination on a route segment with a longest distance is performed repeatedly until the total distance of the resulting delivery route becomes smaller than the total walkable distance depending on the weather.

According to the present embodiment, as described above, in a bad weather, the total walkable distance is set to be shorter than in a case other than the bad weather, and a destination is deleted such that the total distance of the delivery route is shorter than the set distance. Thus, an increase in the burden on the delivery person due to a bad weather can be suppressed, and the safety for the delivery person can be ensured.

### Embodiment 12

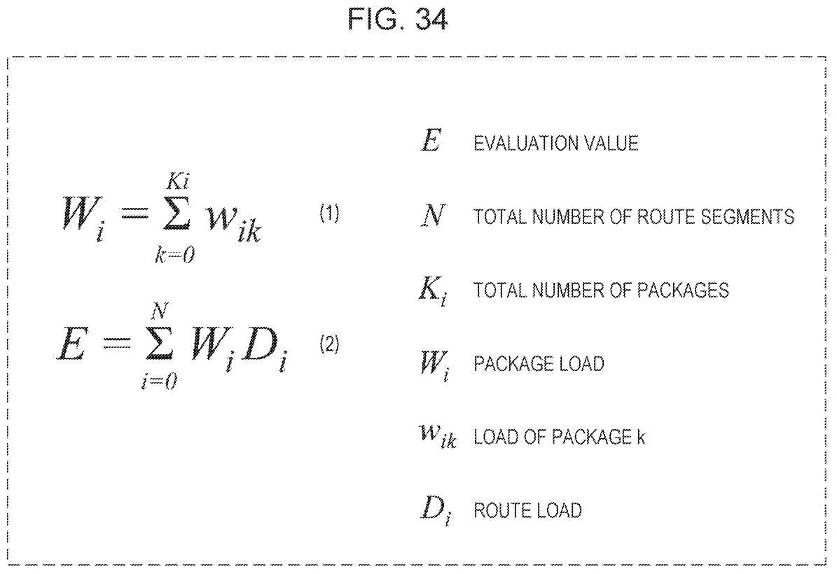

Embodiment 12 provides a specific method of calculating the evaluation value of a delivery route. FIG. 34 shows formulas for calculating the evaluation value. The evaluation value of a delivery route may be calculated according to a formula (2). Wi in formula (2) is given by formula (1). The calculation of the evaluation value according to formulas (1) and (2) is performed by the evaluation value calculator 12.

In formula (2), E denotes the evaluation value. Wi denotes the package load depending on the weight of the package carried by the delivery person during the movement along the i-th route segment of the segments forming the delivery route. Di denotes the route segment load dependent on the distance or the required time for the i-th route segment. For example, when the distance of the i-th route segment is 100 m, the route segment load Di is 100. In a case where the time required for the delivery person to walk over the i-th route segment is 5 minutes, the route segment load Di is 5. N denotes the total number of route segments included in a delivery route. In the example of the delivery route illustrated in FIG. 6, the total number of route segments is 3.

That is, the evaluation value E given by formula (2) indicates the sum total of the delivery loads (=Wi·Di) each obtained by multiplying the package load Wi by the route segment load Di over all route segments.

In formula (1), wik denotes a load (for example, a weight) of a package k carried by the delivery person during the movement on the i-th route segment. Ki denotes the total number of packages carried by the delivery person during the movement along the i-th route segment. Thus, the package load Wi in formula (1) indicates the sum total of packages carried by the delivery person during the i-th route segment.

Thus, the evaluation value E increases as the length of each route segment of the delivery route increases and as the weight of the packages carried along each route segment increases. Thus, by determining the delivery route that provides a minimum evaluation value E as the optimum delivery route, it is possible to present a delivery route results in a low physical burden on the delivery person. The evaluation value E is determined taking into account the package load Wi of the packages carried by the delivery person on foot, that is the evaluation value E accurately reflects the burden on the delivery person.

### Embodiment 13

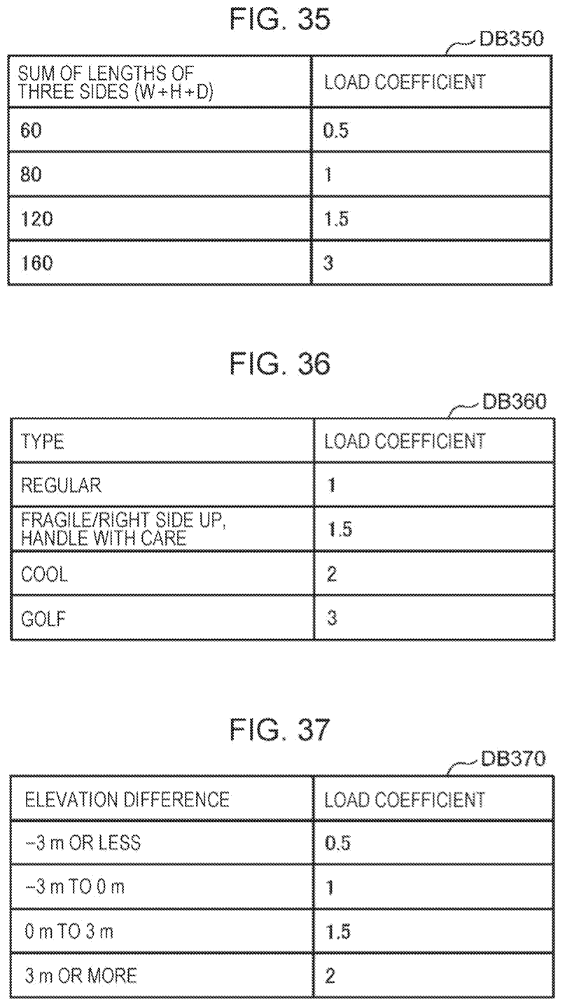

In Embodiment 13, the evaluation value is calculated taking into account sizes of packages. FIG. 35 is a diagram illustrating an example of a data structure of a size-dependent load coefficient DB 350 stored in the memory 11 of the server 1 according to Embodiment 13. The size-dependent load coefficient DB 350 is a database that stores load coefficients corresponding to various package sizes. More specifically, a total value of three sides (W+H+D) and a load coefficient are stored in association with each other. The “total value of three sides (W+H+D)” is the total value of the width (W), the height (H), and the depth (D) of the package, and represents the size of the package. The “load coefficient” is a coefficient used in calculating wik in formula (1). The set value of the load coefficient increases as the size of the package increases. The load coefficient is an example of the first load coefficient.

For example, when the total value of the three sides described in the size-dependent load coefficient DB 350 is equal to or smaller than 60, the evaluation value calculator 12 may select 0.5 as the load coefficient. When the total value of the three sides is greater than 60 and equal to or smaller than 80, for example, 1 may be selected as the load coefficient. When the total value of the three sides is greater than 80 and equal to or smaller than 120, for example, 1.5 may be selected as the load coefficient. When the total value of the three sides is greater than 120 and equal to or smaller than 160, for example, 3.0 may be selected as the load coefficient. When the total value of the three sides is greater than 160, the evaluation value calculator 12 may select, for example, 3.0 as the load coefficient.

A specific example of the calculation of the evaluation value according to formula (1) is described below for case where a package k has a weight of a kg and a size of 60. In this case, the load coefficient is determined as 0.5 from the size-dependent load coefficient DB 350, and wik is calculated as wik=α×0.5. For packages other than the package k, wik can be calculated in a similar manner, that is, wik is given by the weight×the correction coefficient. Then, the evaluation value calculator 12 substitutes the wik calculated for each package into formula (1) thereby obtaining the package load Wi, and calculates the evaluation value E using formula (2).

According to the present embodiment, as described above, the evaluation value is increased as the size of the package is increased, and thus it is possible to calculate the evaluation value more properly taking into account the actual load on the delivery person in the delivery.

### Embodiment 14

In Embodiment 14, the evaluation value is calculated taking into account types of packages. FIG. 36 is a diagram illustrating an example of a data structure of a type-dependent load coefficient DB 360 stored in the memory 11 of the server 1 according to Embodiment 14. The type-dependent load coefficient DB 360 is a database that stores load coefficients corresponding to various package types. More specifically, a “type” and a “load coefficient” are stored in association with each other. The “type” indicates the type of a package, such as “regular”, “fragile/right side up, handle with care”, “cool”, and “golf”. The type “fragile/right side up, handle with care” refers to a package type such as glass, ceramics, and precision machinery that are likely to be broken when turned upside down or thrown. The type “cool” refers to a package type that needs to be cooled, such as fresh food and frozen food. The type “golf” refers to a package type such as a golf bag that stores golf clubs. The type “regular” refers to a package type other than the types described above.

The “load coefficient” is a coefficient used in calculating wik in formula (1). A larger value is assigned as the load coefficient to a package type which is difficult to carry or requires a nerve to handle and thus which is a heavy burden for a delivery person to carry. Note that this load coefficient is an example of the second load coefficient.

A specific example of the calculation of the evaluation value according to formula (1) is described below for a case where a package k has a weight of a kg and a cool type. In this case, the load coefficient is determined as 2 from the type-dependent load coefficient DB 360, and wik is calculated as wik=α×2. For packages other than the package k, wik can be calculated in a similar manner, that is, wik is given by the weight×the correction coefficient. Then, the evaluation value calculator 12 substitutes the wik calculated for each package into formula (1) thereby obtaining the package load Wi, and calculates the evaluation value E using formula (2).

According to the present embodiment, as described above, the evaluation value is properly determined taking into account the type-dependent delivery load on the delivery person.

### Embodiment 15

In Embodiment 15, the evaluation value is calculated taking into account an elevation difference in each route segment of a delivery route. FIG. 37 is a diagram illustrating an example of a data structure of an elevation difference-dependent load coefficient DB 370 stored in the memory 11 of the server 1 according to Embodiment 15. The elevation difference-dependent load coefficient DB 370 is a database that stores load coefficients corresponding to various elevation differences. More specifically, an “elevation difference” and a “load coefficient” are stored in association with each other. The “elevation difference” refers to an elevation difference of an i-th route segment. Note that the elevation difference for an i-th route segment may be given by a difference between a maximum elevation and a minimum elevation of the i-th route segment. Alternatively, the elevation difference for the i-th route segment may be given by a sum total of differences between maximum elevations and minimum elevations calculated for respective uphill and downhill sections included in the i-th route segment. Note that the elevation difference has a positive value for any uphill slope and a negative value for any downhill slope. The elevation difference of each route segment may be stored in advance in the route segment length DB 51 and the route segment length DB 52 illustrated in FIG. 5.

The “load coefficient” is a coefficient used in calculating the route segment load Di in formula (2). The set value of the load coefficient increases as the uphill elevation difference increases. Note that this load coefficient is an example of the third load coefficient.

For example, the evaluation value calculator 12 may select the value of the load coefficient from values stored in the elevation difference-dependent load coefficient DB 370 as follows: 0.5 is selected as the load coefficient when the elevation difference is equal to or smaller than −3 m; 1 is selected when the elevation difference is greater than −3 m and equal to or smaller than 0 m; 1.5 is selected when the elevation difference is greater than 0 m and equal to or smaller than 3 m; and 2 is selected when the elevation difference is greater than 3 m.

A specific example of the calculation of the evaluation value according to formula (2) is described below for a case where an i-th route segment has a length of 100 m and an elevation difference of 3 m. In this case, the load coefficient is determined as 1.5 from the elevation difference-dependent load coefficient DB 370, and the route segment load Di is calculated as Di=100×1.5. Then, the evaluation value calculator 12 substitutes the route segment loads Di of the respective route segments into formula (2) thereby calculating the evaluation value E. Note that in the present embodiment, the package loads Wi may be calculated according to one of the methods disclosed in Embodiments 12 to 14.

According to the present embodiment, as described above, the evaluation value of the delivery route increases with increasing number of route segments having an uphill elevation difference and with increasing uphill elevation difference. and thus the actual burden on the delivery person is properly taken into account in the calculation of the evaluation value.

### Embodiment 16

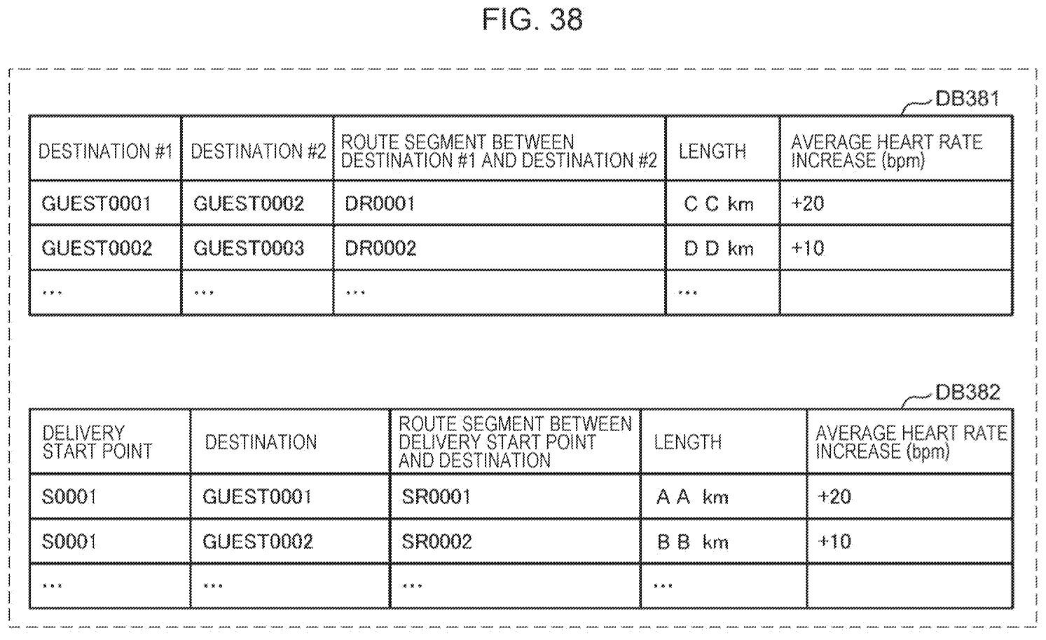

In Embodiment 16, the evaluation value is calculated taking into account an average increase rate of a heart rate of the delivery person on each of route segments forming a delivery route. FIG. 38 is a diagram illustrating an example of a data structure of a route segment length DB 381 and a route segment length DB 382 stored in the memory 11 of the server 1 according to Embodiment 16. The route segment length DB 381 and the route segment length DB 382 each further include a field of “average heart rate increase rate” in addition to the fields of the route segment length DB 51 and the route segment length DB 52 illustrated in FIG. 5.

The “average heart rate increase rate” is calculated by measuring the heart rate with a sensor when a delivery person carries packages along each route segment. More specifically, on each route segment, the difference between the heart rate at the start of delivery and the heart rate at the arrival at the destination is divided by the time required for travel, and the average value taken for all delivery persons is determined.

In order to realize this, it is assumed that the delivery person wears a sensor for measuring the heart rate during delivery. The delivery person terminal 2 may periodically transmit the heart rate measured using this sensor to the server 1 in association with the measurement time and the current position. The control unit 15 of the server 1 may store the heart rate, the measurement time, and the current position transmitted from the delivery person terminal 2 in association with each other in the memory 11 as a heart rate log. Then, the control unit 15 may calculate, as required, the average heart rate increase rate for each route segment length by referring to the heart rate log and may store it in the route segment length DB 381 and the route segment length DB 382.

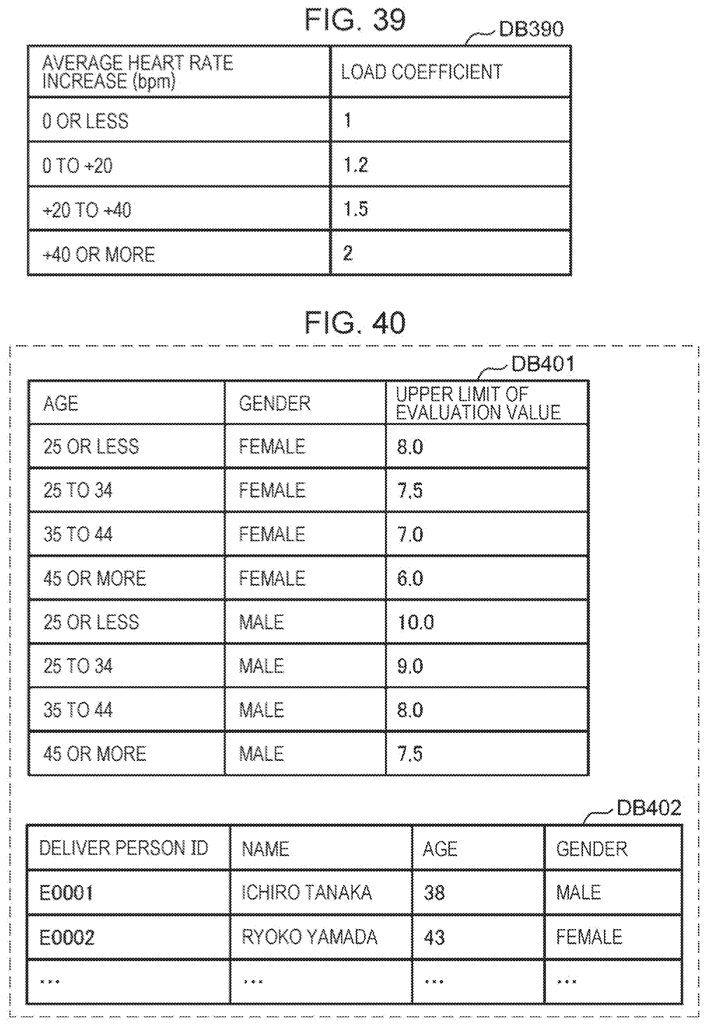

FIG. 39 is a diagram illustrating an example of a data structure of a heart rate-dependent load coefficient DB 390 according to Embodiment 16. The heart rate-dependent load coefficient DB 390 is a database that stores a load coefficient depending on an average heart rate increase rate, and the “average heart rate increase rate” and the “load coefficient” are stored in association with each other.

The “average heart rate increase rate” corresponds to the “average heart rate increase rate” stored in the route segment length DB 381 and the route segment length DB 382. The “load coefficient” is a coefficient used in calculating the route segment load Di in formula (2). The set value of the load coefficient increases as the average heart rate increase rate increases. Note that this load coefficient is an example of the fourth load coefficient.

For example, the evaluation value calculator 12 may select the value of the load coefficient from values stored in the heart rate-dependent load coefficient DB 390 as follows: 1 is selected as the load coefficient when the average heart rate increase rate is smaller than 0; 1.2 is selected when the average heart rate increase rate is greater than 0 and equal to or smaller than 20; 1.5 is selected when the average heart rate increase rate is greater than 20 and equal to or smaller than 40; and 2 is selected when the average heart rate increase rate is greater than 40.

A specific example of the calculation of the evaluation value according to formula (2) is described below for a case where an i-th route segment has a length of 100 m and an average heart rate increase rate of 20. In this case, the load coefficient is determined as 1.2 from the heart rate-dependent load coefficient DB 390, and the route segment load Di is calculated as Di=100×1.2. Then, the evaluation value calculator 12 substitutes the route segment loads Di of the respective route segments into formula (2) thereby calculating the evaluation value E. Note that in the present embodiment, the package loads Wi may be calculated according to one of the methods disclosed in Embodiments 12 to 14.

In the present embodiment, the evaluation value of the delivery route increases with increasing number of route segments in which a large average increase rate of heart rate occurs and with increasing average increase rate of the heart rate, and thus it is possible to properly take into account the actual burden on the delivery person in the calculation of the evaluation value.

In the present embodiment, the delivery route is calculated taking into account the rate of increase in the heart rate of the delivery person on the delivery route. In general, delivering after the delivery vehicle is stopped is performed two or more times a day. Therefore, when it is found that the delivery load along a certain delivery route is high due to an occurrence of an increase in the heart rate or the like, it is possible to increase the load coefficient in a next-time calculation of the delivery route. This makes it possible to select a delivery route taking into account the degree of fatigue due to the delivery work, which may increase in the afternoon.

### Embodiment 17

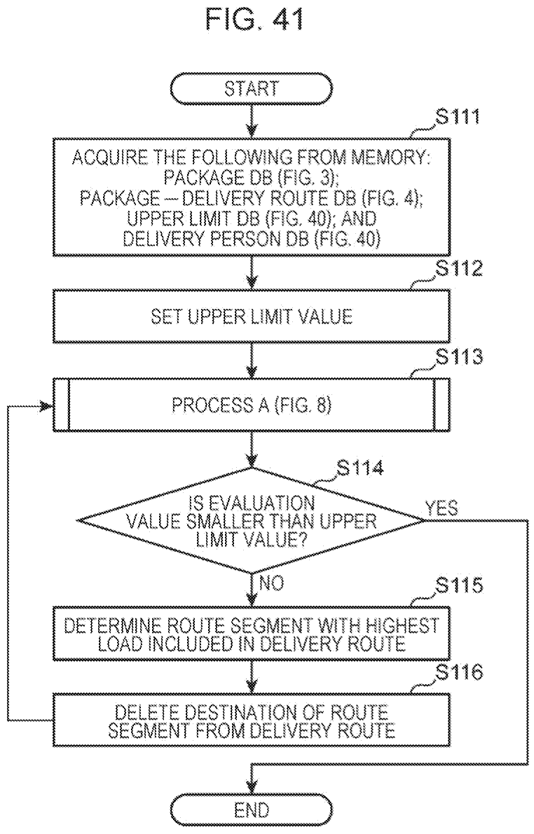

In Embodiment 17, the delivery route is calculated taking into account an age of a delivery person. FIG. 40 is a diagram illustrating an example of a data structure of an upper limit DB 401 and a delivery person DB 402 stored in the memory 11 of the server 1 according to Embodiment 17. The upper limit DB 401 is a database that stores an upper limit evaluation value of a delivery route depending on the age of the delivery person, and an “age”, a “gender”, and an “upper limit evaluation value” are stored in association with each other. The “age” indicates the age of the delivery person. The “gender” indicates the gender of the delivery person. The “upper limit evaluation value” indicates the upper limit of the evaluation value for the delivery route. Note that the upper limit evaluation value is set so as to decrease as the age increases for the same gender. For the same age, the upper limit evaluation value is set such that a smaller value is set for women than for men. This is because the physical strength of the delivery person decreases as the age increases, and women have less physical strength than men. The ages may be classified into a class of 25 or younger, a class of age older than 25 and equal to or younger than 34, a class of age older than 34 and equal to or younger than 44, and so on, and the upper limit of the evaluation value is set for each class. Note that the classification described above is merely an example.

The delivery person DB 402 is a database that stores personal information of the delivery person, and a “delivery person ID”, a “name”, an “age”, and a “gender” are stored in association with each other. The “delivery person ID” is an identifier of the delivery person. The “name” indicates the name of the delivery person. The “age” indicates the age of the delivery person. The “gender” indicates the gender of the delivery person.

FIG. 41 is a flowchart illustrating an example of a process performed by the information providing system according to Embodiment 17. In S111, the control unit 15 acquires the package DB 31, the package-delivery route DB 41, the upper limit DB 401, and the delivery person DB 402 from the memory 11.

In S112, the control unit 15 identifies the age and the gender of the delivery person by referring to the delivery person DB 402, and sets the upper limit evaluation value corresponding to the age and the gender of the delivery person by referring to the upper limit DB 401.

More specifically, in S112, the control unit 15 may refer to the delivery person DB 402 using, as a key, information transmitted from a delivery person terminal 2 as to a delivery person ID of a delivery person having the delivery person terminal 2, thereby identifying an “age”, and a “gender” of the delivery person. For example, as a result of referring to the delivery person DB 402, the delivery person may be identified as a man of an age of 38. In this case, the upper limit DB 401 is referred to, and as a result, 8.0 is set as the upper limit of the evaluation value.

In S113, the process A is executed. As a result, a delivery route is determined. Note that, in the process A, S2 to S4 illustrated in FIG. 8 are executed. In S114, the control unit 15 determines whether or not the evaluation value of the delivery route determined in S113 is smaller than the upper limit of the evaluation value set in S112. In a case where the evaluation value is smaller than the upper limit of the evaluation value (YES in S114), the delivery route determined in S113 has an appropriate load for the corresponding delivery person, and thus this delivery route is determined as the optimum delivery route, and the process is ended.

On the other hand, in a case where the evaluation value is equal to or greater than the upper limit of the evaluation value (NO in S114), the process proceeds to S115. In S115, the control unit 15 identifies a route segment having the largest load in the route segments forming the delivery route determined in S113. Here, as the load, the package load Wi in formula (2) may be used, or the route segment load Di or the delivery load given by Wi×Di may be used. In the example of the delivery route illustrated in FIG. 6, the delivery route is composed of the three route segments “SR0001”, “DR0001”, and “SR0002”, and thus a route segment with the largest load is identified from these route segments.

In S116, the control unit 15 deletes one of destinations located at upstream and downstream nodes of the largest-load route segment identified in S115 from the delivery route determined in S113. The details of this process are the same as the process in S109 in FIG. 33. That is, in the processing flow illustrated in FIG. 41, deleting of a destination on a route segment with a largest load is performed repeatedly until the evaluation value of the resulting delivery route becomes smaller than the upper limit of the evaluation value corresponding to the age and the gender of the delivery person.

As described above, according to the present embodiment, the upper limit of the evaluation value is selected depending on the age and the gender, and in the case where the evaluation value of the delivery route is equal to or larger than the upper limit of the evaluation value, a destination is deleted such that the resultant evaluation value of the delivery route becomes smaller than the upper limit of the evaluation value. Thus, the delivery route that is proper in terms of the load on the delivery person is presented.

In the present embodiment, information on factors such as the age and/or the gender related to the load on the delivery person is input, and the delivery route is selected based on that information. However, the physical condition of the delivery person can change daily. Therefore, the delivery person may input his/her current physical condition such as “good physical condition”, “normal physical condition”, “poor physical condition”, or the like before he/she starts the delivery, and the delivery route may be calculated taking into account the physical condition of the delivery person.

The physical condition of the delivery person may change even in one day. For example, when the delivery route was selected under the condition of “good physical condition” in the morning, if his/her physical condition becomes bad in the afternoon, the physical condition may be changed to “poor physical condition”, and the delivery route may be selected taking into account the changed physical condition. This makes it possible to calculate the delivery route taking into account the physical condition of the delivery person, which may change in a short time.

### Embodiment 18

Embodiment 18 is similar to Embodiment 13 described above except that using a trolley by a delivery person in carrying packages is further taken into account in calculating evaluation values.

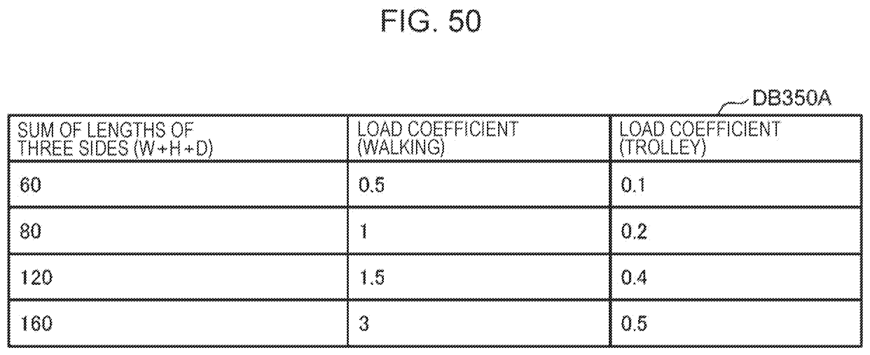

FIG. 50 is a diagram illustrating an example of a data structure of a size-dependent load coefficient DB 350A stored in the memory 11 of the server 1 according to Embodiment 18. The size-dependent load coefficient DB 350A includes fields of “load coefficient (walking)” and “load coefficient (trolley)” instead of the field of “load coefficient” in the size-dependent load coefficient DB 350 illustrated in FIG. 35. The “load coefficient (walking)” is a load coefficient applied when the delivery person carries packages on foot, and is the same as the “load coefficient” in the size-dependent load coefficient DB 350. The “load coefficient (trolley)” is a load coefficient applied when the delivery person carries packages using a trolley.

The trolley has a plate portion on which packages are placed, rollers disposed on a back surface of the plate portion, a handle portion that extends upward from the plate portion for use the delivery person to push the trolley. The trolley is used for collectively carrying packages taken out from a delivery vehicle to destinations. The handle portion of the trolley is rotatably attached to the plate portion, and the trolley is configured to be foldable. When moving by the delivery vehicle, the trolley is folded and loaded onto the delivery vehicle.

The trolley may have an assist function for assisting the delivery person in pushing the trolley with a motor or the like. The trolley may be a cart. The cart may be a tower type trolley in which grid-shaped side walls extends upward from an edge of the plate portion.

The physical load burdened on the delivery person when carrying packages on a trolley is smaller than when carrying packages on foot. Therefore, in the size-dependent load coefficient DB 350A, when the sizes of packages are the same, the value of the “load coefficient (trolley)” is set to be smaller than the value of the “load coefficient (walking)”.

The “load coefficient (walking)” and the “load coefficient (trolley)” are coefficients used in calculating wik in formula (1). These load coefficients are set so as to increase as the size of the package increases.

For example, the evaluation value calculator 12 may select the value of the load coefficient (trolley) from values stored in the size-dependent load coefficient DB 350A as follows: 0.1 is selected as the load coefficient (trolley) when the sum of three sides is equal to or smaller than 60; 0.2 is selected as the load coefficient (trolley) when the sum of three sides is larger than 60 and equal to or smaller than 80; 0.4 is selected as the load coefficient (trolley) when the sum of three sides is larger than 80 and equal to or smaller than 120; and 0.5 is selected as the load coefficient (trolley) when the sum of three sides is larger than 120 and equal to or smaller than 160. In a case where the sum of three sides is larger than 160, the evaluation value calculator 12 may select, for example, 0.5 as the load coefficient (trolley).

A specific example of the calculation according to formula (1) is described below for a case where a package k having a weight of α kg and a size of 60 is carried using a trolley. In this case, the load coefficient (trolley) is determined as 0.1 from the size-dependent load coefficient DB 350A, and thus wik is calculated as wik=α×0.1. In a case where the package k is carried on foot, the load coefficient (walking) is determined as 0.5 from the size-dependent load coefficient DB 350A, and thus wik is calculated as wik=α×0.5. Then, the evaluation value calculator 12 substitutes the wik calculated for each package into formula (1) thereby obtaining the package load Wi along an i-th route segment, and calculates the evaluation value E by substituting the load coefficient Wi into formula (2).

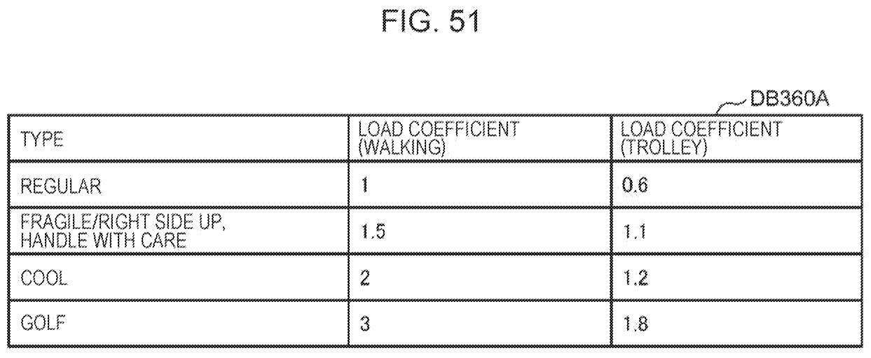

In the present embodiment, wik may be calculated using the type-dependent load coefficient DB 360A illustrated in FIG. 51 instead of the size-dependent load coefficient DB 350A.

FIG. 51 is a diagram illustrating an example of a data structure of a type-dependent load coefficient DB 360A stored in the memory 11 of the server 1 according to Embodiment 18. The type-dependent load coefficient DB 360A includes fields of “load coefficient (walking)” and “load coefficient (trolley)” instead of the field of “load coefficient” in the type-dependent load coefficient DB 360 illustrated in FIG. 36. The “load coefficient (walking)” is a load coefficient applied when the delivery person carries packages on foot, and is the same as the “load coefficient” in the type-dependent load coefficient 360. The “load coefficient (trolley)” is a load coefficient applied when the delivery person carries packages using a trolley.

The physical load burdened on the delivery person when carrying packages on a trolley is smaller than when carrying packages on foot. Therefore, in the type-dependent load coefficient DB 360A, when the types of packages are the same, the value of the “load coefficient (trolley)” is set to be smaller than the value of the “load coefficient (walking)”.

The “load coefficient (walking)” and the (load coefficient (trolley)” are coefficients used in calculating wik in formula (1). A larger value is set for a package type which causes a heavy burden on a delivery person to carry, for example, as in a case where it is difficult to carry the package or requires a nerve to handle.

A specific example of the calculation according to formula (1) is described below for a case where a package k having a weight of αkg and a type of “cool” is carried using a trolley. In this case, the load coefficient (trolley) is determined as 1.2 from the type-dependent load coefficient DB 360A, and thus wik is calculated as wik=α×1.2. On the other hand, when the package k is carried on foot, the load coefficient (walking) is 2, and thus wik is calculated by wik=α×2. Then, the evaluation value calculator 12 substitutes the wik calculated for each package into formula (1) thereby obtaining the package load Wi along an i-th route segment, and calculates the evaluation value E by substituting the load coefficient Wi into formula (2).

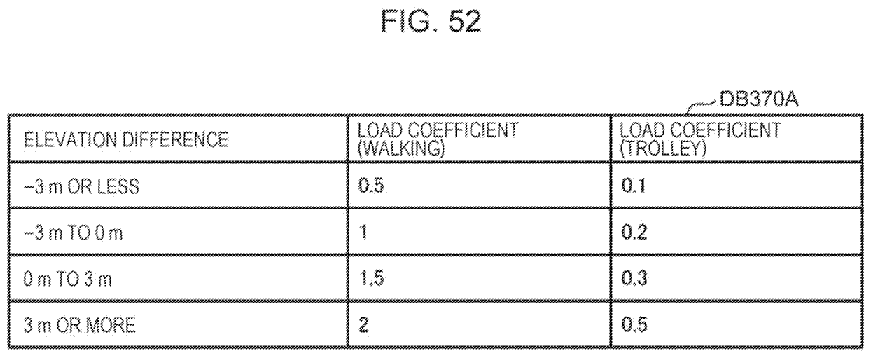

In the present embodiment, wik may be calculated using the size-dependent load coefficient DB 350A or the type-dependent load coefficient DB 360A, and, using this wik, the route segment load Di may be calculated based on the elevation difference-dependent load coefficient DB 370A illustrated in FIG. 52 and the further calculation of the evaluation value E may be performed.

FIG. 52 is a diagram illustrating an example of a data structure of an elevation difference-dependent load coefficient DB 370A stored in the memory 11 of the server 1 according to Embodiment 18. The elevation difference-dependent load coefficient DB 370A includes fields of “load coefficient (walking)” and “load coefficient (trolley)” instead of the field of “load coefficient” in the elevation difference-dependent load coefficient DB 370 illustrated in FIG. 37. The “load coefficient (walking)” is a load coefficient applied when the delivery person carries packages on foot, and is the same as the “load coefficient” in the elevation difference-dependent load coefficient DB 370. The “load coefficient (trolley)” is a load coefficient applied when the delivery person carries packages using a trolley.

The physical load burdened on the delivery person when carrying packages on a trolley is smaller than when carrying packages on foot. Therefore, in the elevation difference-dependent load coefficient DB 370A, when the elevation differences are the same, the value of the “load coefficient (trolley)” is set to be smaller than the value of the “load coefficient (walking)”.

For example, when the delivery person carries packages on a trolley, the evaluation value calculator 12 may select the value of the load coefficient from values stored in the elevation difference-dependent load coefficient DB 370A as follows: 0.1 is selected as the load coefficient when the elevation difference is equal to or smaller than −3 m; 0.2 is selected when the elevation difference is greater than −3 m and equal to or smaller than 0 m; 0.3 is selected when the elevation difference is greater than 0 m and equal to or smaller than 3 m; and 0.5 is selected when the elevation difference is greater than 3 m.

A specific example of the calculation of the evaluation value according to formula (2) is described below for a case where a package is carried using a trolley along an i-th route segment having length of 100 m and an elevation difference of 3 m. In this case, the load coefficient (trolley) is determined as 0.3 from the elevation difference-dependent load coefficient DB 370A, and the route segment load Di is calculated as Di=100×0.3. In contrast, when the packages are carried on foot along the i-th route segment, the load coefficient (walking) is 1.5, and thus the route segment load Di is calculated as Di=100×1.5. Then, the evaluation value calculator 12 substitutes the route segment loads Di of the respective route segments into formula (2) thereby calculating the evaluation value E.

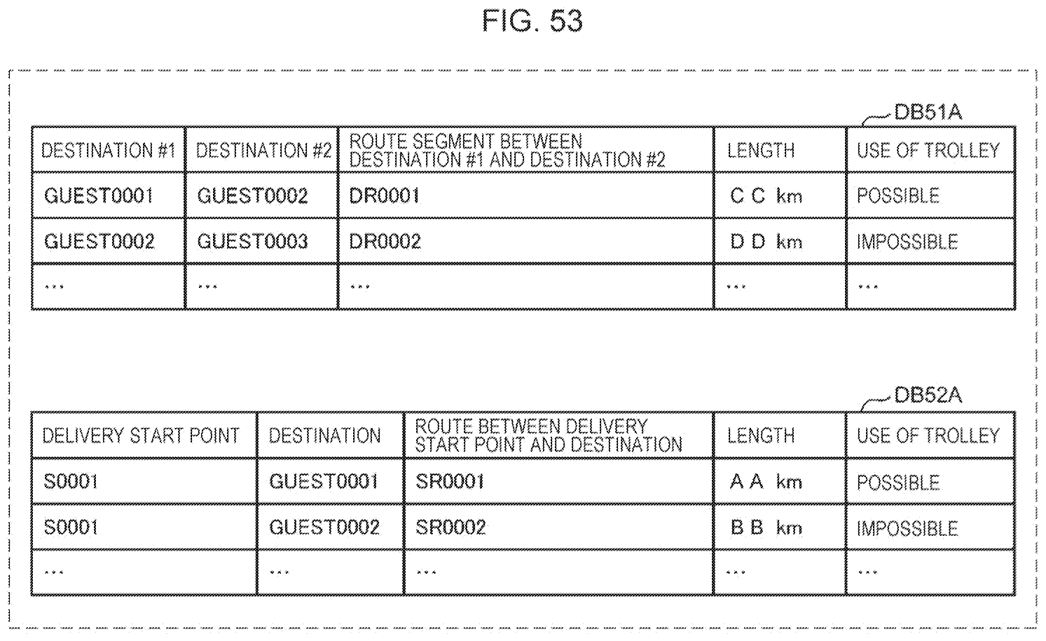

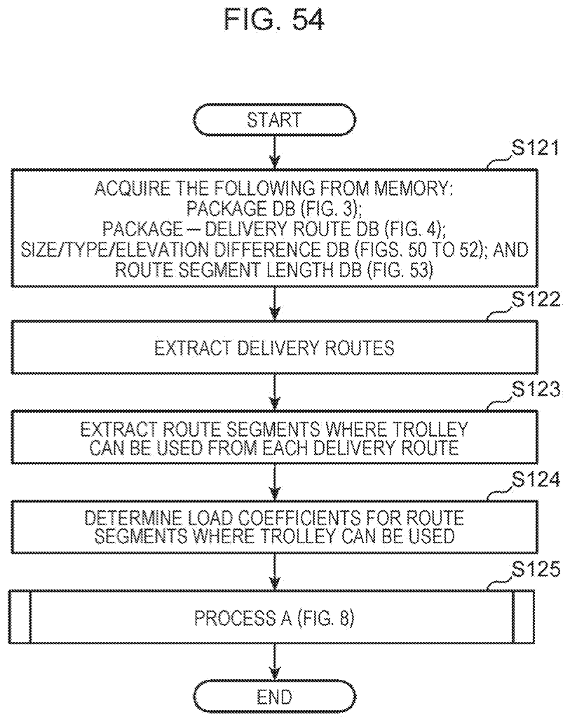

In the present embodiment, the route segment length of an i-th route segment is calculated using the route segment length DB 51A and the route segment length DB 52A illustrated in FIG. 53 instead of the route segment length DB 51 and the route segment length DB 52 illustrated in FIG. 5.

FIG. 53 is a diagram illustrating an example of a data structure of a route segment length DB 51A and a route segment length DB 52A according to Embodiment 18. The route segment length DB 51A is a database for storing information related to route segments connecting destinations on a delivery route indicated by delivery route information stored in the delivery route DB 42. The route segment length DB 51A further includes a field of “use of trolley” in addition to the fields of the route segment length DB 51 illustrated in FIG. 5. “Use of trolley” indicates whether or not it is possible to use a trolley on a corresponding route segment. For example, a trolley is usable on a route segment “DR0001” described in a first row, “possible” is described in the field of “use of trolley”. However, a trolley is not usable on a route segment “DR0002” described in a second row, “impossible” is described in the field of “use of trolley”.

The route segment length DB 52A is a database for storing information related to a route segment connecting a delivery start point and a destination on a delivery route indicated by delivery route information stored in the delivery route DB 42. The route segment length DB 52A further includes a field of “use of trolley” in addition to the fields of the route segment length DB 52 illustrated in FIG. 5. Information described in the field of “use of trolley” indicates whether or not it is possible to use a trolley on a corresponding route segment. For example, whether a trolley is usable on a route segment “SR0001” described in a first row, “possible” is described in the field of “use of trolley”. However, a trolley is not usable on a route segment “SR0002” described in a second row, “impossible” is described in the field of “use of trolley”.

For example, when an i-th route segment is “DR0001”, using of a trolley is possible, and thus, wik in formula (1) is calculated using the value of the “load coefficient (trolley)” stored in the size-dependent load coefficient DB 350A or the type-dependent load coefficient DB 360A, and the route segment load Di in the formula (2) is calculated using the value of the “load coefficient (trolley)” stored in the elevation difference-dependent load coefficient DB.

Examples of route segments where a trolley cannot be used include a route segment whose width is narrower than the width of the trolley, a route segment where the ground is soft and thus it is difficult to move the trolley, a route segment including stairs, a route segment which is steep, which makes it difficult to move the trolley, etc.

FIG. 54 is a flowchart illustrating an example of a process performed by the information providing system according to Embodiment 18. In S121, the control unit 15 of the server 1 acquires, from the memory 11, the package DB31, the package-delivery route DB 41, the size-dependent load coefficient DB 350A or the type-dependent load coefficient DB 360A, the elevation difference-dependent load coefficient DB 370A, the route segment length DB 51A, and the route segment length DB 52A.

In S122, the evaluation value calculator 12 extracts delivery routes corresponding to packages to be collectively delivered by the delivery person on foot or by a trolley. More specifically, in this case, package IDs corresponding to the packages to be delivered are identified from the package group DB 43, and furthermore a delivery route ID corresponding to the package IDs is identified from the package-delivery route DB 41, and delivery routes corresponding to the delivery route ID are identified from the delivery route DB 42.

In S123, the evaluation value calculator 12 refers to the route segment length DB 51A and the route segment length DB 52A, and extracts route segments where the trolley is usable for each delivery route. A further explanation is given for a case where one of the extracted delivery routes is that illustrated in FIG. 6, and to this delivery route, the route segment length DB 51A and the route segment length DB 52A are applied. In this case, the trolley can be used for route segments “SR0001” and “DR0001”, but the trolley cannot be used for a route segment “SR0002”, and thus the route segments “SR0001” and “DR0001” are extracted.

In S124, the evaluation value calculator 12 refers to the size-dependent load coefficient DB 350A or the type-dependent load coefficient DB 360A and determines the load coefficients for the route segments where the trolley can be used. In the example of the delivery route in FIG. 6, the value of the “load coefficient (trolley)” is acquired for each of the route segments “SR0001” and “DR0001” by referring to the size-dependent load coefficient DB 350A or the type-dependent load coefficient DB 360A. Furthermore, the value of the “load coefficient (trolley)” is acquired by referring to the elevation difference-dependent load coefficient DB 370A.

In S125, the process A is executed on the delivery routes extracted in S122 in which the load coefficients determined in S124 are applied. In the process A, S2 to S4 illustrated in FIG. 8 are executed. However, in this specific example, since delivery routes are already extracted in S122, the process of extracting delivery routes in S2 is omitted, and the evaluation value is calculated for each of the delivery routes extracted in S122. In S4, the delivery route is output together with information indicating whether the trolley is usable for each route segment of the delivery route. The delivery route output here may be a delivery route in which the trolley can be used in all route segments thereof, a delivery route in which the trolley can be used in part of the route segments, or a delivery route in which the trolley cannot be used in any of route segments.

Although no description has been given above as to handling to be performed when the delivery route includes a route segment where the trolley cannot be used, the evaluation value may be calculated by handling the trolley as described below.

For example, let it be assumed that in the example of the delivery route illustrated in FIG. 6, the trolley can be used in the route segments “SR0001” and “SR0002”, but the trolley cannot be used in the route segment “DR0001”. In this case, when the delivery of the package at the destination “GUEST0001” is completed, the delivery person leaves the trolley at this place and carries the remaining package on foot to the destination “GUEST0002”. After the delivery at the destination “GUEST0002” is completed, the delivery person returns to the destination “GUEST0001” to pick up the trolley, and then goes to the delivery start point “S0001” via the route segment “SR0001”. Thus, in this case, the delivery route sequentially passes through the first route segment “SR0001”, the second route segment “DR0001”, the third routes “DR0001”, and the fourth route segment “SR0001”.

The evaluation value in this case is estimated as follows. The package load W1 and the route segment load D1 on the first route segment “SR0001” are calculated using the package loads and the route segment loads which are applied when all packages are carried using the trolley. The package load W2 and the route segment load D2 on the second route segment “DR0001” are calculated using the package loads and the route segment loads which are applied when the remaining packages are carried on foot. The package load W3 and the route segment load D3 on the third route segment “DR0001” are calculated using the package load and the route segment load which are applied when the remaining package is carried on foot. In a case where there is no package to be carried along the third route segment load, the package load and the route segment load for the package bag on foot are used. The package load W4 and the route segment load D4 on the fourth route segment “SR0001” are calculated using the package load and the route segment load which are applied when the remaining package is carried using the trolley. In a case where there is no package to be carried along this route segment load, the package load and the route segment load for the package bag using the trolley are used.

In the example described above, the size-dependent load coefficient DB 350A and the type-dependent load coefficient DB 360A are used alternatively. However, the present embodiment is not limited to this, and both the size-dependent load coefficient DB 350A and the type-dependent load coefficient DB 360A may be used. In this case, wik illustrated in formula (1) may be calculated as follows. For example, let it be assumed that a package k is carried using a trolley where the sum of W+H+D of the package k is 60, the type of the package k is cool, and the weight of the package k is a kg. In this case, as for the load coefficient (trolley), 0.1 is acquired from the size-dependent load coefficient DB 350A, and 1.2 is acquired from the type-dependent load coefficient DB 360A. Then, wik may be calculated as wik=α×(0.1×1.2) or wik=α×(0.1+1.2).

In the present embodiment, as described above, since the evaluation values are calculated taking into account whether the trolley is usable in carrying packages, the evaluation value is calculated taking into account the physical load on the delivery person more accurately. Furthermore, in the present embodiment, the delivery person is presented with information on the delivery route including information indicating in which route segment the trolley can be used use and in which route segment the trolley cannot be used, which makes it possible for the delivery person to properly use the trolley to efficiently carry the packages.

### Embodiment 19

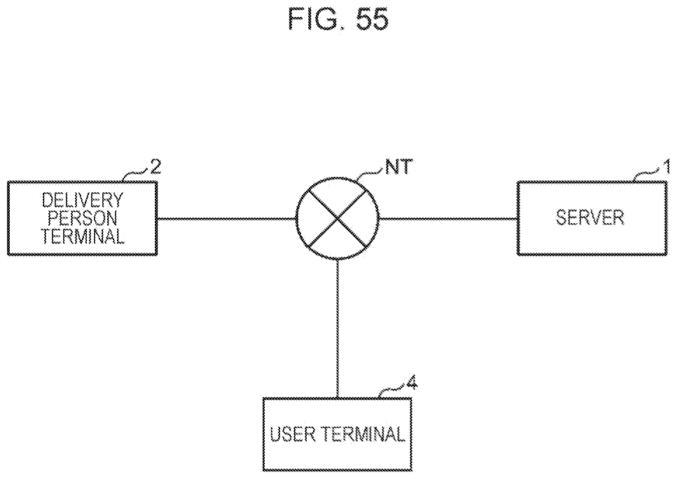

In Embodiment 19, when a delivery person is heading to a first destination included in the destinations, information indicating that the delivery person is heading is notified to the recipient at the first destination. FIG. 55 is a diagram illustrating an example of a network configuration of an information providing system according to Embodiment 19. In this embodiment, a user terminal 4 (an example of the second information terminal) is added to the information providing system illustrated in FIG. 1.

The user terminal 4 is a terminal used by a recipient of a package. The user terminal 4 may be a portable information terminal such as a smartphone, a portable telephone device, a tablet terminal, or the like, or may be a stationary information processing terminal.

In the present embodiment, when the delivery person terminal gets off the delivery vehicle to carry a package, the server 1 acquires information indicating this fact from the delivery person terminal 2 via a network NT. More specifically, for example, the delivery person terminal 2 acquires position information indicating the position of the delivery vehicle from the GPS sensor disposed on the delivery vehicle. When the position information indicates that the delivery vehicle has been stopped for a predetermined period or longer, it is determined that the delivery person has got off the delivery vehicle, and the delivery person terminal 2 transmits a getting-off signal to the server 1. Alternatively, for example, instead of or in addition to responding to the position information of the GPS sensor indicating that the delivery vehicle is stopped, in response to acquiring, from the delivery vehicle, a signal indicating that the engine of the delivery vehicle is stopped, the delivery person terminal 2 may transmit the getting-off signal. Alternatively, instead of or in addition to responding to detecting an occurrence of the above-described condition, in response to acquiring, from the delivery vehicle, a signal indicating that a door of the delivery vehicle is opened or closed, the delivery person terminal 2 may transmit the getting-off signal.

When the control unit 15 of the server 1 acquires the getting-off signal transmitted from the delivery person terminal 2, the control unit 15 identifies one or more recipients of packages to be delivered by the delivery person after this delivery person gets off the delivery vehicle, and the control unit 15 transmits, to the user terminal 4 of the identified recipient via the communicator 13, information indicating that the delivery person is heading to the recipient.

In a case where the delivery vehicle is stopped at a predetermined delivery start point and packages are delivered as in Embodiment 2 described above, the control unit 15 may extract packages to be delivered to destinations whose distance from the delivery start point is equal to smaller than a threshold value from the package DB 31 and the customer DB 32, and the control unit 15 may identify the recipients of the extracted packages from the customer DB 32.

In a case where the delivery person does not know the delivery start point as in Embodiment 3 described above, the control unit 15 may identify recipients as follows. First, when the control unit 15 detects that the delivery vehicle has stopped based on information given from the delivery person terminal 2, the control unit 15 guides the delivery vehicle to a predetermined delivery start point as in Embodiment 3. Next, when the control unit 15 detects that the delivery vehicle has reached the delivery start point and acquires the getting-off signal from the delivery person terminal 2, the control unit 15 identifies the recipients using the above-described method.

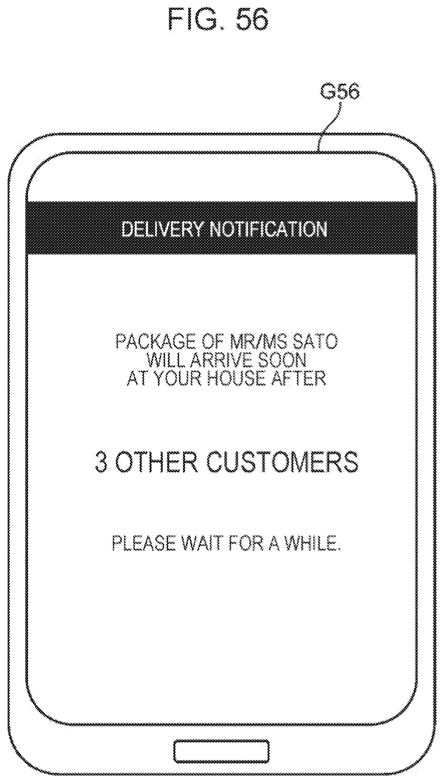

FIG. 56 is a diagram illustrating an example of a notification screen G56 displayed on a user terminal 4 to inform that a delivery vehicle is heading to a recipient according to Embodiment 19. On the notification screen G56, a heading “DELIVERY NOTIFICATION” is displayed. Below the heading is a message that the package will be delivered soon. More specifically, in this example, the displayed message is “PACKAGE OF MR/MS SATO WILL ARRIVE SOON AT YOUR HOUSE AFTER 3 OTHER CUSTOMERS. PLEASE WAIT FOR A WHILE”.

As described above, this message contains information indicating how many packages will been delivered before the package arrives at the recipient. This makes it possible for the recipient to estimate an approximate time for which the recipient has to wait before the delivery person visits his/her home. Thus, the recipient can prepare to receive the package, such that the recipient can receive the package more reliably.

As described above, according to the present embodiment, when the delivery person gets off the delivery vehicle, information is transmitted to the user terminal 4 of the package recipient to notify that the delivery person is heading to the recipient, and thus it is possible to prevent the recipient from being absent when the package arrives at the recipient.

### Specific Examples of Delivery Routes

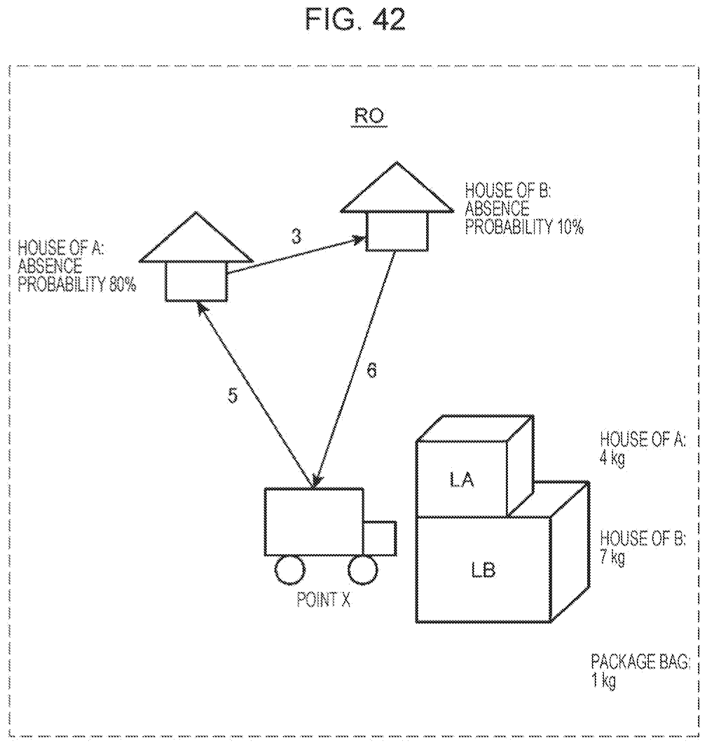

Next, specific examples of delivery routes are described below. FIG. 42 is a diagram illustrating an example of a delivery route RO. In this example, the delivery route RO starts from a point X at a delivery start point, passes through a house of A at a destination and further a house of B at a next destination, ant finally returns to the point X.

A package LA with a weight of 4 kg is delivered to the house of A, and a package LB with a weight of 7 kg is delivered to the house of B. In this delivering, the delivery person puts the package LA and the package LB in a package bag with a weight of 1 kg, and carries the package bag on foot. The absence probability of a recipient at the house of A is 80%, and the absence probability of a recipient at the house of B is 10%. The movement cost from the X point to the house of A is 5, the movement cost from the house of A to the house of B is 3, and the moving cost from the house of B to the point X is 6. Note that the movement cost indicates the load on the delivery person taking into account the carried packages, and is represented, for example, by a route segment load Di in formula (2).

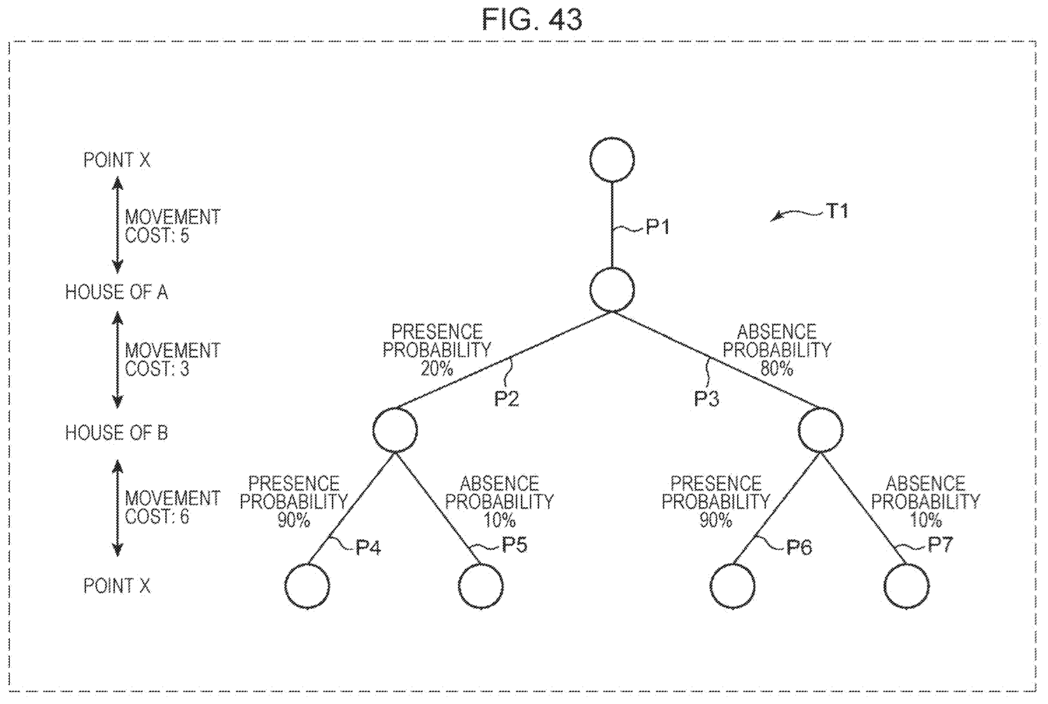

FIG. 43 is a probability binary tree representation T1 of the delivery route illustrated in FIG. 42. This binary tree T1 includes seven paths P1 to P7, where the path P1 connects from the point X to the house of A, the path P2 connects from the house of A to the house of B when a recipient is present at the house of A, the path P3 connects from the house of A to the house of B when the recipient is absent at the house of A, and so on.

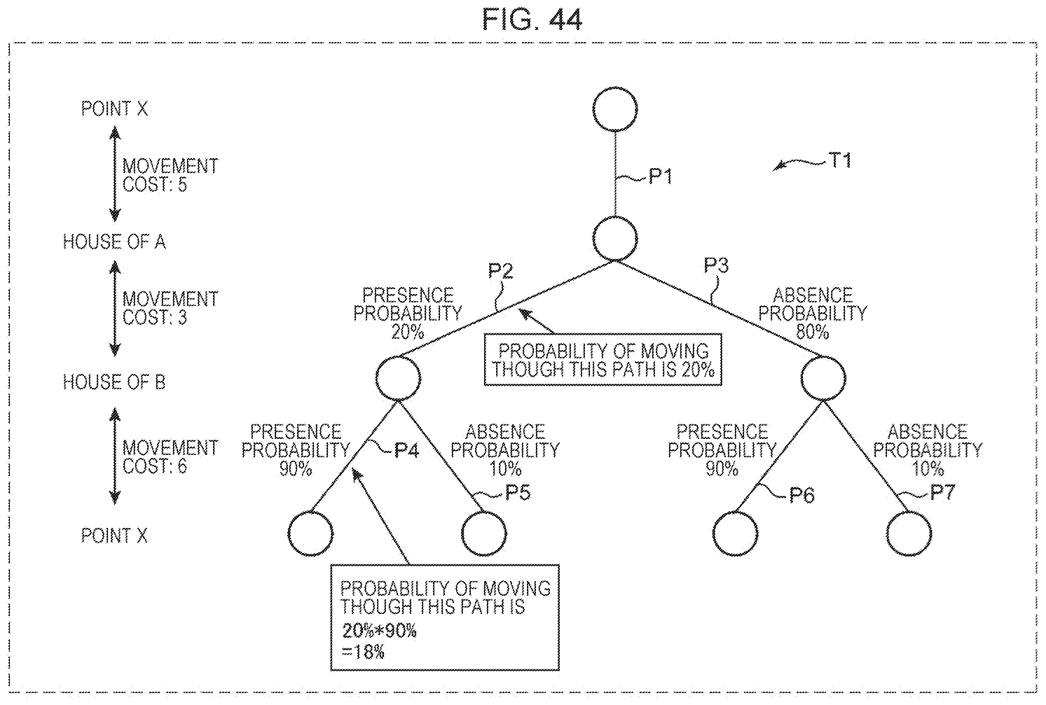

FIG. 44 is a diagram further illustrating the binary tree T1 illustrated in FIG. 43. As illustrated in FIG. 44, for example, since the absence probability of the house of A is 80%, the probability that the delivery person passes through the path P2 is 20%, and the probability that the delivery passes through the path P3 is 80%. The absence probability of the house of B is 10%, and thus the probability that the delivery person passes through the path P4 is given by the probability 20% of the path P2 times the probability 90% of the path P4, that is, 18%. In this way, the probability that the delivery person passes through each path is calculated.

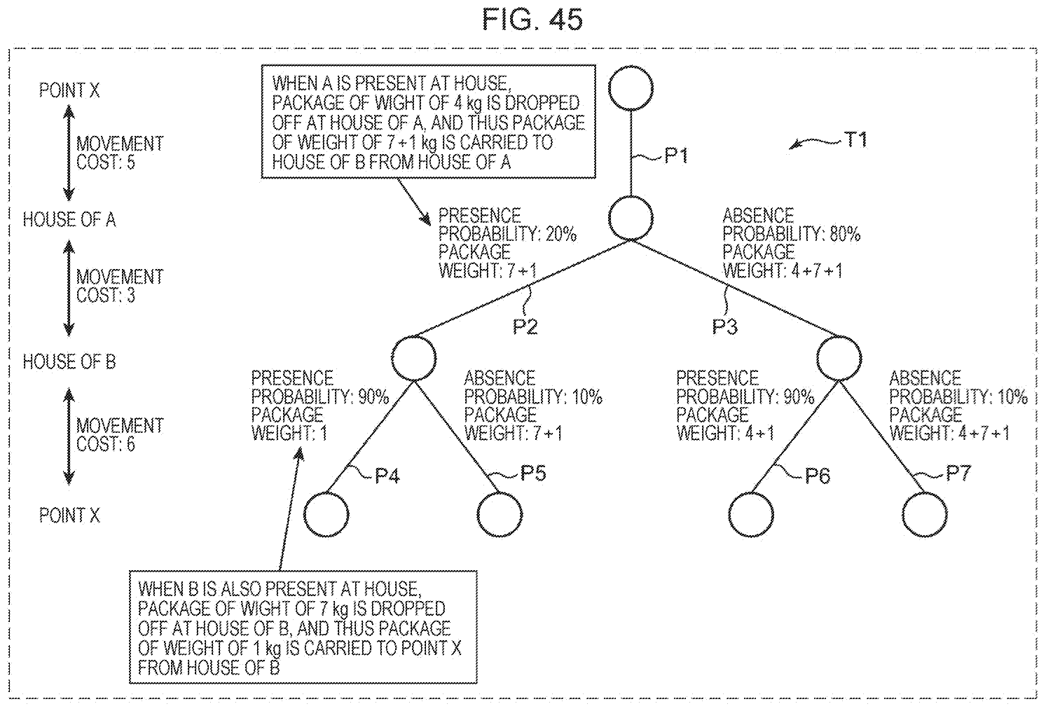

FIG. 45 is a diagram further illustrating the binary tree T1 illustrated in FIG. 44. In a case where A is present at the house of A, the package LA of the weight of 4 kg is dropped off at the house of A, and thus the weight of the packages carried by the delivery person on the pass P2 is given by the sum of the weight of the package LB, 7 kg, and the weight of the package bag, 1 kg, that is, 8 kg. On the other hand, when A is absent at the house of A, a package LA is not dropped off at the house of A, and thus the weight of the packages carried by the delivery person on the pass P3 is given by the sum of the weights of the package LA, the package LB, and the package bag, that is, 4 kg+7 kg+1 kg=12 kg. In the pass P4, since the packages were dropped off at both the house of A and the house of B, the delivery person carries only the package bag of the weight of 1 kg. In this way, the probability that the delivery person passes a path and the weight of the packages carried by the delivery person on the path are calculated for each of the paths P1 to P7.

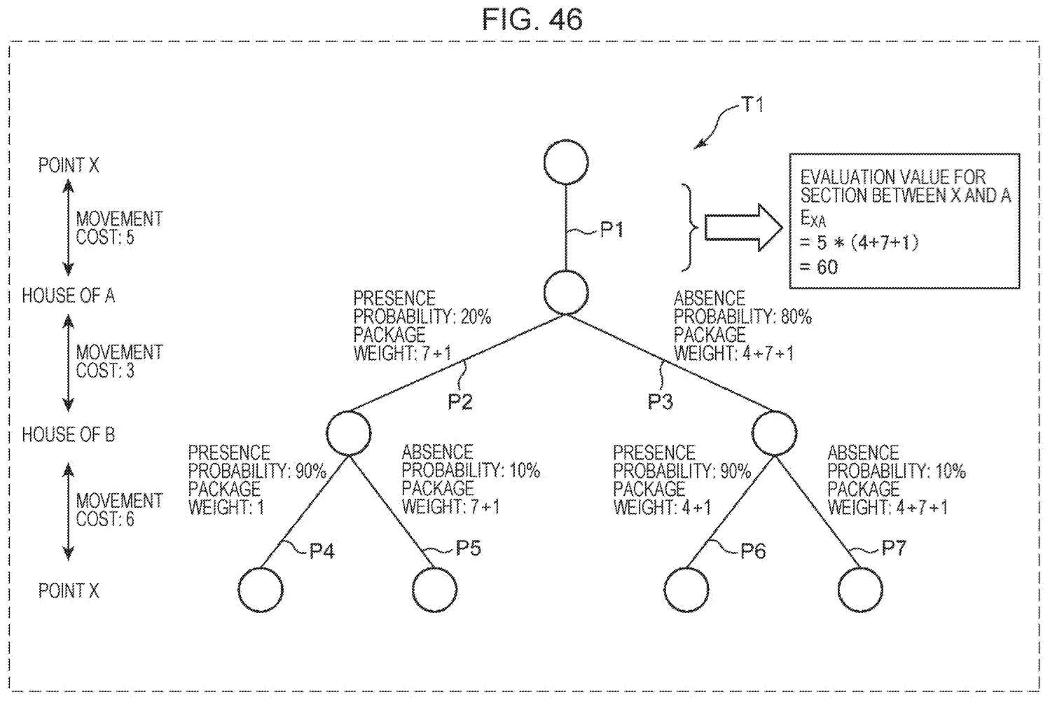

FIG. 46 is a diagram further illustrating the binary tree T1 illustrated in FIG. 45. With reference to this figure, an explanation is given here as to the evaluation value EXA for the path P1 between the point X and the house of A. The weight of packages carried by the delivery person between the point X and the A house is given by the sum of the weights of the package LA, the package LB, and the package bag, that is, 4 kg+7 kg+1 kg. The movement cost between the point X the house of A is 5. Thus, the evaluation value EXA is given as EXA=5×(4+7+1)=60.

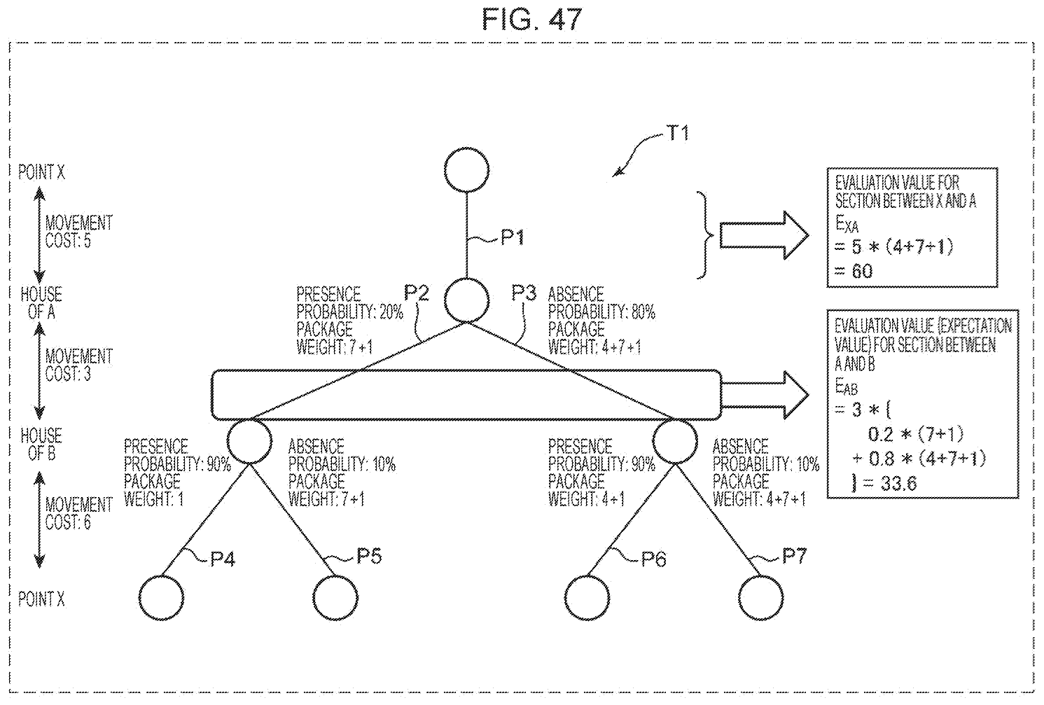

FIG. 47 is a diagram further illustrating the binary tree T1 illustrated in FIG. 46. With reference to this figure, an explanation is given here as to the evaluation value EAB between the house of A and the house of B. In the pass P2, the weight of the packages carried by the delivery person is given by the sum of 7 kg for the package LB and 1 kg for the package bag. In the pass P3, the weight of the packages carried by the delivery person is given by the sum of 4 kg for the package LA and 1 kg for the package bag. The movement cost between the house of A and the house of B is 3. Thus, the evaluation value EAB is given as EAB=3×{0.2×(7+1)+0.8×(4+7+1)}=33.6.

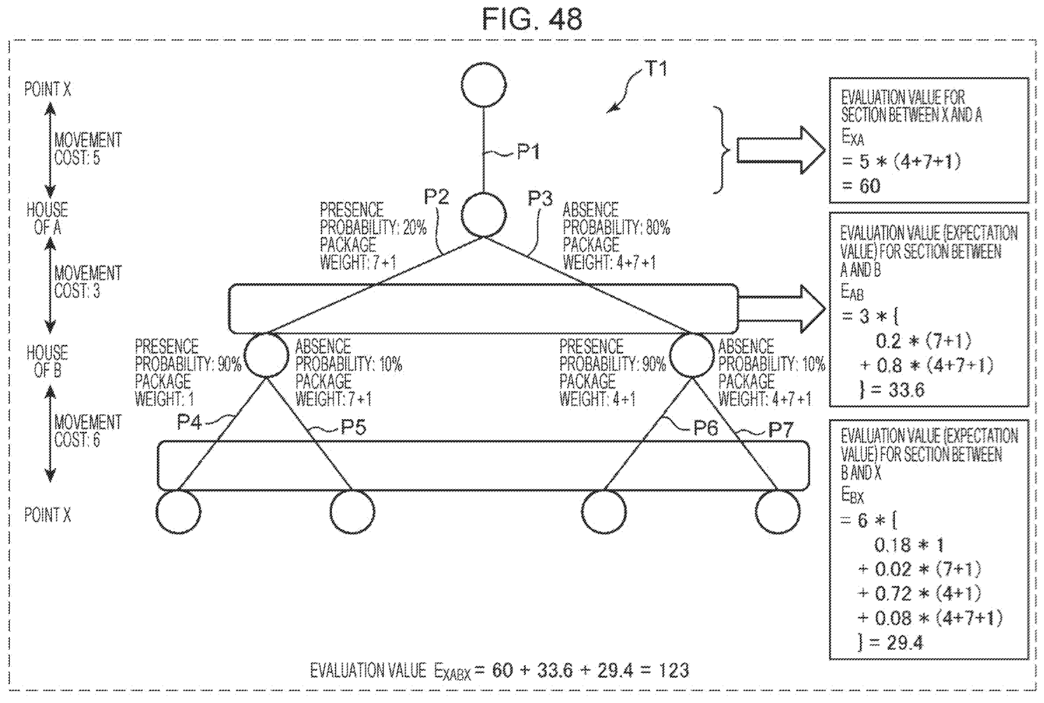

FIG. 48 is a diagram further illustrating the binary tree T1 illustrated in FIG. 47. With reference to this figure, an explanation is given here as to the evaluation value EBX between the house of B and the point X. In the pass P4, the delivery person carries only the package bag of the weight of 1 kg. In the pass P5, the weight of the packages carried by the delivery person is given by the sum of 7 kg for the package LB and 1 kg for the package bag. In the pass P6, the weight of the packages carried by the delivery person is given by the sum of 4 kg for the package LA and 1 kg for the package bag. In the pass P7, the weight of the packages carried by the delivery person is given by the sum of 4 kg for the package LA, 7 kg for the package LB, and 1 kg for the package bag. The movement cost between the house of B and the point X is 6. Thus, the evaluation value EBX is given as EBX=6×{(0.18×1+0.02×(7+1)×0.72×(4+1)+0.08×(4+7+1)}=29.4 Thus, the evaluation value EXABX for he delivery route RO is given as 60+33.6+29.4=123.

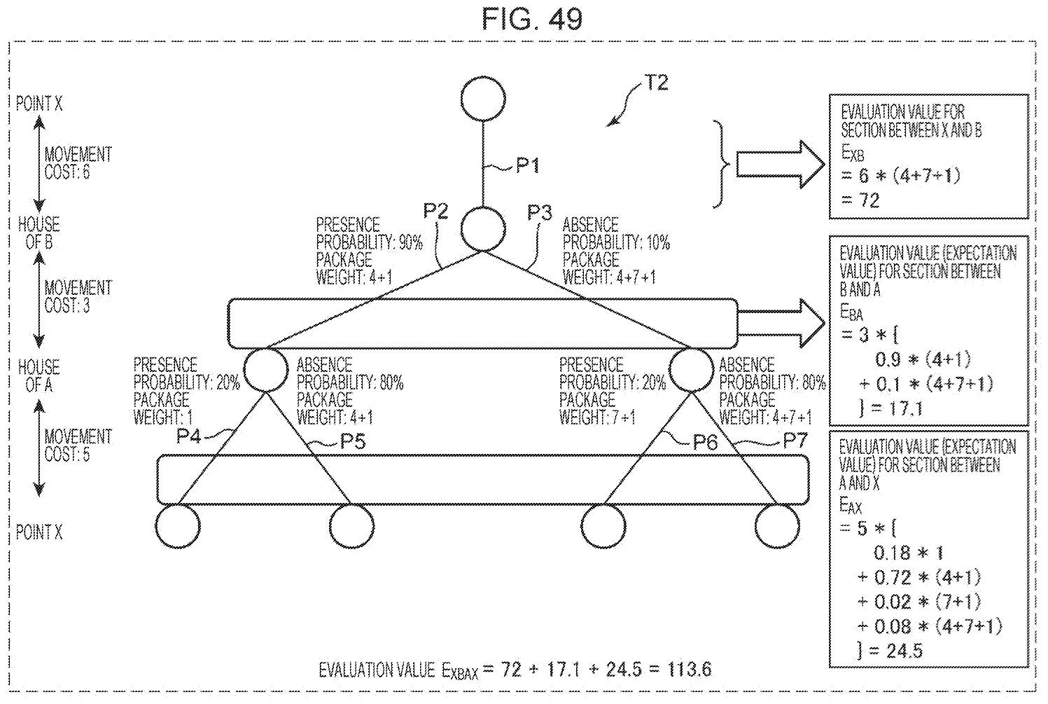

FIG. 49 is a probability binary tree representation T2 of a delivery route R1 different from that illustrated in FIG. 42. The delivery route R1 is a delivery route obtained by exchanging the delivery order between the house of A and the house of B in the delivery route RO illustrated in FIG. 42. As a result of the exchange of the delivery order between the house of A and the house of B, the weights of the packages carried by the delivery person on the respective paths P1 to P7 and the probabilities that the delivery person passes through the respective paths P1 to P2 are different between the probability binary tree T2 and the probability binary tree Ti. However, the weights of packages and the probabilities can be calculated in a similar manner to the probability binary tree T1.

That is, the evaluation values EXB, EBA, and EAX are respectively calculated as EXA=72, EBA=17.1, and EAX=24.5. Then, the evaluation value XBAX of the delivery route RO is calculated as 72+17.1+24.5=113.6.

Although both delivery routes pass through the same destinations, the comparison between the above results for the delivery route RO and the delivery route R1, indicates that the evaluation value EXBAX of the delivery route R1 is smaller than the evaluation value EXABX of the delivery route RO, that is, it is indicated that the delivery route R1 provides a less burden on the delivery person than the delivery route RO. Thus, in this specific case, the delivery route R1 is determined as the optimum delivery route and is notified to the delivery person.

### (Modifications)

In the embodiments described above, the optimum delivery route is determined using the evaluation values, but the present disclosure is not limited to this, and other indicators may be used to determine the optimum delivery route.

The elevation difference stored in the elevation difference-dependent load coefficient DB 370 may be treated as information indicating the road condition included in the first delivery route information.

According to the present disclosure, it is possible to present an efficient delivery route along which a delivery person may carry packages on foot, which is useful for improving the efficiency of the delivery work of the delivery person.

What is claimed is:

1. An information providing method comprising, by a computer in an information providing system,
acquiring, from a memory, first package information indicating a size or a weight of each of packages and first delivery route information indicating first delivery routes, wherein each of the first delivery routes starts from a delivery start point, passes through destinations to which the packages are to be delivered, and ends at the delivery start point;
calculating, based on the first package information and the first delivery route information, an evaluation value for each of the first delivery routes;
determining, based on the calculated evaluation values, an optimum first delivery route from among the first delivery routes; and
outputting, to a first information terminal, information indicating the determined first delivery route,
wherein the determined first delivery route is displayed on a display of the first information terminal, and
wherein a delivery person delivers the packages to the destinations along the determined first delivery route.

2. The information providing method according to claim 1, wherein
each evaluation value represents a physical load on the delivery person.

3. The information providing method according to claim 1, wherein
each evaluation value reflects a fact that each time the delivery person delivers a package to a destination, a sum of sizes or weights of remaining ones of the packages becomes smaller.

4. The information providing method according to claim 1, wherein
each evaluation value is calculated as a sum total of delivery loads on route segments including route segments connecting the delivery start point and the destinations when the delivery start point and the destinations are connected serially and route segments between the destinations,
wherein the delivery load on an i-th route segment (i is an integer equal to or greater 0) is represented by a product of a package load depending on weight of one or more packages to be delivered on the i-th route segment and a route segment load depending on a distance or a traveling time on the i-th route segment.

5. The information providing method according to claim 4, wherein
the first delivery route information includes at least one of followings: distances of the first delivery routes; travel times of the first delivery routes; and road conditions of the first delivery routes.

6. The information providing method according to claim 4, wherein
the package load is represented by a sum total of values each of which is obtained by multiplying a weight of each package delivered by the delivery person on the i-th route segment and a first load coefficient of the package, and
the first load coefficient is set so as to increase as the size of the package increases.

7. The information providing method according to claim 6, wherein
the package load varies depending on whether or not a trolley is used.

8. The information providing method according to claim 7, wherein
the first delivery route information includes information regarding whether the trolley is usable.

9. The information providing method according to claim 8, wherein
the first delivery route information includes a first route segment on which the delivery person uses the trolley and a second route segment on which the delivery person does not use the trolley.

10. The information providing method according to claim 4, wherein
the package load is represented by a sum total of values each of which is obtained by multiplying a weight of each package carried by the delivery person on the i-th route segment and a second load coefficient of the package, and
the second load coefficient is set to a value according to a type of the package.

11. The information providing method according to claim 4, wherein
the first delivery route information includes a distance of each of the route segments and an elevation difference of each of the route segments, and
the route segment load is represented by a sum total of values each of which is obtained by multiplying the distance of the i-th route segment and a third load coefficient, and
the third load coefficient is set so as to increase as the elevation difference increases in an uphill direction.

12. The information providing method according to claim 4, wherein
the first delivery route information includes a length of each of the route segments and an average increase rate of a heart rate of the delivery person on each of the route segments,
the route segment load is represented by a sum total of values each of which is obtained by multiplying the length of an i-th route segment and a fourth load coefficient, and
the fourth load coefficient is set so as to increase as the average increase rate of the heart rate increases.

13. The information providing method according to claim 1, wherein
the first package information includes a delivery destination of each of the packages,
delivery destinations whose distances from the delivery start point are equal to or less than a threshold value are extracted, based on the first package information, from the delivery destinations, and
each evaluation value is calculated for each of the first delivery routes including the extracted delivery destinations as the destinations.

14. The information providing method according to claim 1, further comprising
further acquiring, from the memory, delivery start point information in which candidates for the delivery start point are stored in association with positions;
further acquiring a current position of the delivery person; and
further determining, as the delivery start point, a candidate for the delivery start point closest to the current position of the delivery person from among the candidates for the delivery start point.

15. The information providing method according to claim 14, wherein
in a case where information indicating that a delivery vehicle of the delivery person is unable to be stopped at the delivery start point is acquired from the first information terminal via a network,
a candidate for the delivery start point next closest to the current position of the delivery person is determined as the delivery start point from among the candidates for the delivery start point.

16. The information providing method according to claim 15, wherein
the information indicating that the delivery vehicle of the delivery person is unable to be stopped at the delivery start point includes a reason why the delivery vehicle is unable to be stopped.

17. The information providing method according to claim 1, wherein
in a case where the delivery vehicle of the delivery person is enabled to be stopped at the delivery start point, information regarding appropriateness of the delivery start point is further acquired from the first information terminal via a network.

18. The information providing method according to claim 1, further comprising,
in a case where, when the delivery person visits a first destination included in the destinations, information indicating that a first recipient at the first destination is absent is acquired from the first information terminal via a network,
acquiring, from the memory, two or more pieces of second delivery route information indicating second delivery routes each of which serially connects remaining destinations starting from the first destination and returns to the delivery start point;
calculating, based on the first package information and the two or more pieces of second delivery route information, an evaluation value for each of the second delivery routes;
determining, based on the calculated evaluation values, an optimum second delivery route from among the second delivery routes;
outputting information indicating the determined second delivery route to the first information terminal; and
causing the determined second delivery route to be displayed on the display of the first information terminal.

19. The information providing method according to claim 1, further comprising
reading, from the memory, history information indicating a redelivery rate in a past at each of the destinations,
wherein the evaluation value of each of the first delivery routes is calculated based on the history information.

20. The information providing method according to claim 1, further comprising,
in a case where information is acquired from first information terminal via a network, the information indicating that when the delivery person visits a first destination included in the destinations, a first package included in the packages is delivered and a second package is picked up,
acquiring second package information regarding a size or a weight of the second package from the first information terminal via a network;
acquiring, from the memory, two or more pieces of second delivery route information indicating second delivery routes each of which serially connects remaining destinations starting from the first destination and returns to the delivery start point;
calculating, based on the first package information, the second package information, and the two or more pieces of second delivery route information, an evaluation value for each of the second delivery routes;
determining, based on the calculated evaluation values, an optimum second delivery route from among the second delivery routes;
outputting information indicating the determined second delivery route to the first information terminal; and
causing the second delivery route to be displayed on the display of the first information terminal.

21. The information providing method according to claim 1, further comprising,
in a case where information is stored in advance in the memory, the information indicating that at a first destination included in the destinations, the delivery person is to deliver a first package included in the packages and pick up a second package,
acquiring, from the memory, second package information regarding a size or a weight of the second package,
wherein the evaluation value of each of the first delivery routes is calculated based on the first package information and the second package information.

22. The information providing method according to claim 1, further comprising
acquiring, from the first information terminal, information indicating that the delivery person gets off a delivery vehicle;
outputting, to a second information terminal owned by a recipient at a first destination included in the destinations, information indicating that the delivery person is heading to the first destination; and
causing the information indicating that the delivery person is heading to the first destination to be displayed on a display of the second information terminal.

23. The information providing method according to claim 1, wherein
the first delivery route information includes a distance of each of the first delivery routes, and
the method further comprises
acquiring, from the memory, an upper limit length information including a first upper limit distance in walking of the delivery person and a second upper limit distance smaller than the first upper limit distance;
in a case where information indicating bad weather is acquired via a network, setting the second upper limit distance; and
in a case where a distance of the determined first delivery route is equal to or more than the second upper limit distance, deleting a destination included in the first delivery route such that the distance of the first delivery route becomes smaller than the second upper limit distance.

24. The information providing method according to claim 1, further comprising
acquiring, from the memory, an upper limit evaluation value corresponding to an age or gender of the delivery person; and
in a case where the evaluation value of the determined first delivery route is equal to or higher than the upper limit evaluation value, deleting a destination included in the first delivery route such that the evaluation value of the first delivery route becomes smaller than the upper limit evaluation value.

25. An information providing method comprising, by a computer in an information providing system,
acquiring, from a memory, first package information indicating a size or a weight of each of packages and first delivery route information indicating first delivery routes, wherein each of the first delivery routes starts from a delivery start point, passes through destinations to which the packages are to be delivered, and ends at the delivery start point;
determining, based on the first package information and the first delivery route information, an optimum first delivery route from among the first delivery routes; and
outputting, to an information terminal, information indicating the determined first delivery route,
wherein the determined first delivery route is displayed on a display of the information terminal, and
wherein a delivery person delivers the packages to the destinations along the determined first delivery route.

26. An information providing system comprising:
a memory storing first package information indicating a size or a weight of each of packages and first delivery route information indicating first delivery routes, wherein each of the first delivery routes starts from a delivery start point, passes through destinations to which the packages are to be delivered, and ends at the delivery start point;
an evaluation value calculator that calculates, based on the first package information and the first delivery route information, an evaluation value for each of the first delivery routes;
a delivery route determiner that determines, based on the calculated evaluation values, an optimum first delivery route from among the first delivery routes; and
a communicator that outputs, to an information terminal, information indicating the determined first delivery route,
wherein the information terminal displays the determined first delivery route on a display, and
a delivery person delivers the packages to the destinations along the determined first delivery route.

27. An information providing system comprising:
a memory storing first package information indicating a size or a weight of each of packages and first delivery route information indicating first delivery routes, wherein each of the first delivery routes starts from a delivery start point, passes through destinations to which the packages are to be delivered, and ends at the delivery start point,
a delivery route determiner that determines, based on the first package information and the first delivery route information, an optimum first delivery route from among the first delivery routes; and
a communicator that outputs, to an information terminal, information indicating the determined first delivery route,
wherein the information terminal displays the determined first delivery route on a display, and
a delivery person delivers the packages to the destinations along the determined first delivery route.
